(19) United States
(12) Patent Application Publication
(10) Pub. No.: US 2024/0082207 A1
(43) Pub. Date: Mar. 14, 2024

(54) MACROPHAGES/MICROGLIA IN NEURO-INFLAMMATION ASSOCIATED WITH NEURODEGENERATIVE DISEASES

(71) Applicant: The General Hospital Corporation, Boston, MA (US)

(72) Inventors: David R. Elmaleh, Newton, MA (US); Rudolph E. Tanzi, Milton, MA (US); Timothy M. Shoup, Franklin, MA (US); Ana Gircluc, Arlington, MA (US)

(21) Appl. No.: 18/210,992

(22) Filed: Jun. 16, 2023

Related U.S. Application Data
(63) Continuation of application No. 17/323,514, filed on May 18, 2021, now Pat. No. 11,679,095.
Application No. 17/323,514 is a continuation of application No. 16/328,956, filed on Feb. 27, 2019, now abandoned.
(60) Provisional application No. 62/382,192, filed on Aug. 31, 2016.

(51) Int. Cl. A61K 31/352; A61K 9/00; A61P 25/28; C07D 311/22; C07D 311/24

(52) U.S. Cl. CPC A61K 31/352; A61K 9/0019; A61K 9/0073; A61P 25/28; C07D 311/22; C07D 311/24; A61K 45/06

(57) ABSTRACT

Described herein are methods of treating neuron inflammation conditions, for example, Alzheimer's disease, Parkinson's disease, Huntington's disease, ischemic stroke, and prion disease, comprising administering a therapeutically effective amount of cromolyn or a cromolyn derivative compound.

### RELATED APPLICATIONS

This application claims the benefit of priority to U.S. Provisional Patent Application Ser. No. 62/382,192, filed Aug. 31, 2016, which is hereby incorporated herein by reference in its entirety.

### FIELD

The invention encompasses methods of treating a neuron inflammation condition comprising administered a therapeutically effective amount to a patient in need thereof of at least one compound having the following formula:

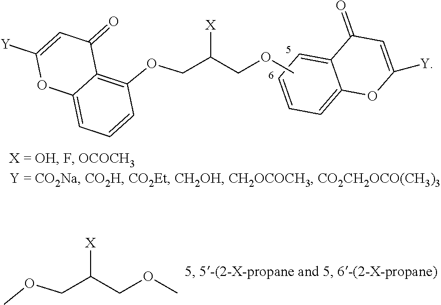

### BACKGROUND

Strategies to modulate monocyte and microglial activity have been studied, especially those that can protect against microglia-mediated neurotoxicity. (See, Zhao et al., “Protective effects of an anti-inflammatory cytokine, interleukin-4, on motoneuron toxicity induced by activated microglia,” J. Neurochem. 2006, 99:1176-1187; Heneka et al., “NLRP3 is activated in Alzheimer's disease and contributes to pathology in APP/PS1 mice,” Nature, 2013, 493(7434):674-8; Theeriault, et al., “The dynamics of monocytes and microglia in Alzheimer's disease,” Alzheimers Res Ther., 2015, 7:41; Nau et al., “Strategies to increase the activity of microglia as efficient protectors of the brain against infections,” Front Cell Neurosci., 2014, 8:138.) Overall, it is clear that more focused studies are needed to better establish how each inflammatory state can modulate the pathology of neurodegenerative diseases such as Alzheimer's Disease (AD) and Amyotrophic Lateral Sclerosis (ALS). Early activation of monocytes and microglia has potential to decelerate neurodegenerative progression by modulating immune responses to increase the intrinsic phagocytic capacity of monocytes and microglia without triggering secretion of pro-inflammatory cytokines that could worsen neurodegeneration.

The role of the neuro-inflammatory response in the presence of amyloid plaques and neurofibrillary tangles in the brain and its associated neuronal loss in the pathology of AD is well established and extensively studied. See, Walker et al., “Immune phenotypes of microglia in human neurodegenerative disease: challenges to detecting microglial polarization in human brains,” Alzheimers Res Ther., 2015, 7:56; Theerialut et al., 2015; Wilcock, D M, “A Changing Perspective on the Role of Neuroinflammation in Alzheimer's Disease,” International Journal of Alzheimer's Disease, 2012, Article ID 495243; McGeer et al., “Targeting microglia for the treatment of Alzheimer's disease,” Expert Opin Ther Targets, 2015, 19(4):497-506). Numerous studies show that microglial-mediated inflammation contributes to the progression of AD and that microglial cells are found in close association with amyloid-β (Aβ) deposits. (See, Mandrekar, et al., “Microglia and Inflammation in Alzheimer's Disease,” CNS Neurol Disord Drug Targets, 2010, 9(2): 156-167).

It is known that the changes in properties of microglia—the brain-resident macrophages—depend on their response to different stimuli in their microenvironment (e.g. cytokines), resulting in a range of phenotypes. Based on the changes in expression of cytokines, receptors, and other markers, monocyte and macrophage states have been defined as: classical activation (M1), alternative activation (M2a), type II alternative activation (M2b), and acquired deactivation (M2c). (See, Walker et al., 2015; Martinez et al., “Alternative activation of macrophages: an immunologic functional perspective,” Annu Rev Immunol. 2009, 27:451-83; Mantovani et al., “The chemokine system in diverse forms of macrophage activation and polarization,” Trends Immunol., 2004, 25:677-686; Sternberg, E M., “Neural regulation of innate immunity: a coordinated nonspecific host response to pathogens,” Nat Rev Immunol., 2006, 6(4):318-28). Recently, a number of studies have attempted to elucidate the role of these phenotypes in the AD brain and determine the mechanisms through which these cells contribute to AD-related neuro-inflammation. (See, Mandrekar et al. 2012; McGeer et al., 2015; and Wilcock, 2012).

Interaction of microglia with fibrillar Aβ leads to their phenotypic activation, and has recently been suggested to play a role in neuroprotection. (See Zhao et al., 2006; Figueiredo et al., “Neuron-microglia crosstalk up-regulates neuronal FGF-2 expression which mediates neuroprotection against excitotoxicity via JNK1/2,” J. Neurochem., 2008 Oct.,107(1):73-85). It has been shown in numerous studies, in both mice and humans, that glial cells respond to the presence of AD pathological lesions (plaques and tangles) by changing their morphological characteristics, expressing numerous cell surface receptors, and surrounding the lesions. (See, Perlmutter et al., “Morphologic association between microglia and senile plaque amyloid in Alzheimer's disease,” Neurosci Lett., 1990, 119:1, 32-36; Combs, et al., “Identification of microglial signal transduction pathways mediating a neurotoxic response to amyloidogenic fragments of (3-amyloid and prion proteins,” J. Neurosci., 1999,19:3, 928-939). On the other hand, macrophage and microglial activation in response to cellular debris in the AD brain, and the subsequent release of pro-inflammatory cytokines, leads to accelerated neurodegeneration. This, in turn, creates more cellular debris and accelerates disease progression. (See, Rubio-Perez et al., “A Review: Inflammatory Process in Alzheimer's Disease, Role of Cytokines,” Scientific World Journal, 2012, 756357; McGeer, et al., “The importance of inflammatory mechanisms in Alzheimer disease,” Exp. Gerontol. 1998, 33:5, 371-378; Akiyama, et al., “Inflammation and Alzheimer's disease,” Neurobiol Aging, 2000, 21(3), 383-421; Liu, et al., “TLR2 is a primary receptor for Alzheimer's amyloid β peptide to trigger neuroinflammatory activation,” J. Immunol. 2012, 188(3):1098-107).

Several studies have focused on microglial activation and its role in the clearance of AD lesions leading to the reduction of amyloid deposits in the brain. (See, DiCarlo, et al., “Intrahippocampal LPS injections reduce Aβ load in APP+PS1 transgenic mice,” Neurobiol of Aging, 2001, 22:6, 1007-1012; Herber, et al., “Time-dependent reduction in Aft levels after intracranial LPS administration in APP transgenic mice,” Exp. Neurol., 2004, 190(1):245-53; Liu, et al., 2012). While resident microglial cells surrounding Aβ plaques are not as efficacious in degrading Aβ as newly infiltrated macrophages or monocytes (See, Thériault, et al., 2015; Varnum, et al., “The classification of microglial activation phenotypes on neurodegeneration and regeneration in Alzheimer's disease brain,” Arch. Immunol. Ther. Exp. (Warsz), 2012, 60(4):251-66), it has been shown that microglia are indeed capable of internalizing fibrillar and soluble Aβ, but are unable to process these peptides. (See Chung, et al., “Uptake, degradation, and release of fibrillar and soluble forms of Alzheimer's amyloid beta-peptide by microglial cells,” J. Biol. Chem., 1999, 274:32301-8).

Further, it has been postulated that microglia undergo a switch from an M2- to an M1-skewed activation phenotype during aging. (See, Heneka et al., 2013; Varnum, et al., 2012; Gratchev, et al., “Mphi1 and Mphi2 can be re-polarized by Th2 or Th1 cytokines, respectively, and respond to exogenous danger signals,” Immunobiology, 2006, 211(6-8):473-486; Colton, et al., “Expression profiles for macrophage alternative activation genes in AD and in mouse models of AD,” J. Neuroinflammation, 2006, 3:27). However, how the immune response in the brain is driven in AD is still a matter of debate, especially whether neuroinflammation can be triggered by age-related systemic inflammation. (See, Thériault, et al., 2015). It has been shown that stimulation of microglia could enhance their intrinsic phagocytic capacity to degrade Aβ more efficaciously; a number of strategies to modulate microglial response have been proposed. (See, Mandrekar, 2010; Kiyota, et al., “CNS expression of anti- inflammatory cytokine interleukin-4 attenuates Alzheimer's disease-like pathogenesis in APP +PS1 bigenic mice,” FASEB J. 2010, 24:3093-3102; He, et al., “Deletion of tumor necrosis factor death receptor inhibits amyloid beta generation and prevents learning and memory deficits in Alzheimer's mice,” J. Cell Biol., 2007, 178:829-841; Varnum, et al., 2012).

It has been shown that microglia are activated by extracellularly deposited Aβ peptide (Lotz, et al., “Amyloid beta peptide 1-40 enhances the action of Toll-like receptor-2 and −4 agonists but antagonizes Toll-like receptor-9-induced inflammation in primary mouse microglial cell cultures,” J. Neurochem., 2005, 94:289-298; Reed-Geaghan, et al., “CD14 and toll-like receptors 2 and 4 are required for fibrillar Aβ-stimulated microglial activation,” J. Neurosci., 2009, 29:11982-11992). This is similar to microglial activation in response to the presence of interferon-γ (IFNγ), tumor necrosis factor alpha (TNFα) from T cells, or antigen-presenting cells. M1 activated microglia can produce reactive oxygen species and result in increased production of pro-inflammatory cytokines such as TNFα and interleukin (IL)-1β.

The M1-type response of microglial cells has been shown to lower amyloid load but exacerbate neurofibrillary tangle pathology. Shaftel et al. (Shaftel, et al., “Sustained hippocampal IL-1β overexpression mediates chronic neuroinflammation and ameliorates Alzheimer plaque pathology,” J. Clin. Invest., 2007, 117(6):1595-604) have shown that IL-1β expression may underlie a beneficial neuroinflammatory response in AD, and that IL-1β overexpression in the hippocampus of APP/PS1 transgenic mice results in decreased amyloid burden. The authors suggest that IL-1β-mediated activation of microglia is the mechanism for the reductions in amyloid deposition. Further, Montgomery et al. (Montgomery, et al., “Ablation of TNF-RI/RII expression in Alzheimer's disease mice leads to an unexpected enhancement of pathology: implications for chronic pan-TNF-α suppressive therapeutic strategies in the brain,” Am. J. Pathol., 2011, 179(4):2053-70) have shown that intact TNF-receptor signaling is critical for microglial-mediated uptake of extracellular amyloid-peptide. While M1 inflammatory phenotypes appear to improve the amyloid pathology in numerous studies, induction of M1 phenotypes in tau transgenic mice or cell culture results in the exacerbation of tau pathology. (See, Kitazawa, et al., “Lipopolysaccharide-induced inflammation exacerbates tau pathology by a cyclin-dependent kinase 5- mediated pathway in a transgenic model of Alzheimer's disease,” J. Neurosci., 2005, 28; 25(39):8843-53.; Li, et al., “Interleukin-1 mediates pathological effects of microglia on tau phosphorylation and on synaptophysin synthesis in cortical neurons through a p38-MAPK pathway,” J. Neurosci., 2003, 1; 23(5):1605-11).

Macrophage M2 activation is associated with mediators that are known to contribute to the anti-inflammatory actions and reorganization of extracellular matrix (Zhu, et al., “Acidic mammalian chitinase in asthmatic Th2 inflammation and IL-13 pathway activation”, Science, 2004, 304(5677):1678-82; Walker, et al., 2015; Wilcock, et al., 2012). Microglia with M2a phenotypes have increased phagocytosis and produce growth factors such as insulin-like growth factor-1 and anti-inflammatory cytokines such as IL-10. Stimulation of macrophages by IL-4 and/or IL-13 results in an M2a state, sometimes called a wound-healing macrophage (Edwards, et al., “Biochemical and functional characterization of three activated macrophage populations,” J. Leukoc Biol., 2006, 80(6):1298-307) and it is generally characterized by low production of pro-inflammatory cytokines (IL-1, TNF and IL-6). The M2a responses are primarily observed in allergic responses, extracellular matrix deposition, and remodeling.

M2b macrophages are unique in that they express high levels of pro-inflammatory cytokines, characteristic of M1 activation, but also express high levels of the anti-inflammatory cytokine IL-10. (See, Moser D M., “The many faces of macrophage activation,” J. Leukoc Biol., 2003, 73(2):209-12).

Finally, the M2c macrophage state is stimulated by IL-10 and is sometimes referred to as a regulatory macrophage. M2c macrophages have anti-inflammatory activity that plays a role in the phagocytosis of cellular debris without the classical pro-inflammatory response (See, Moser D M., 2003). These cells express TGFβ and high IL-10 as well as matrix proteins. (See, Mantovani, et al., “The chemokine system in diverse forms of macrophage activation and polarization,” Trends Immunol., 2004, 25:677-686; Wilcock, et al., 2012). Plunkett et al. (Plunkett, et al., “Effects of interleukin-10 (IL-10) on pain behavior and gene expression following excitotoxic spinal cord injury in the rat,” Exp. Neurol., 2001; 168:144-154) reported that IL-10 mediated anti-inflammatory responses including decreasing glial activation and production of pro-inflammatory cytokines.

However, the mechanism of M2 microglial activation and role it plays in AD and plaque pathology is still not well understood. (See, Mandrekar, et al., 2010). Further, a number of studies suggested that there is a switch in microglial activation status in response to disease progression (Colton, et al., 2006; Jimenez, et al., “Inflammatory response in the hippocampus of PS1M146L/APP751SL mouse model of Alzheimer's disease: age-dependent switch in the microglial phenotype from alternative to classic,” J. Neurosci., 2008, 28:11650-11661). It has been reported in animal studies that microglial activation phenotypes switch from M2 to M1 during disease progression (Jimenez, et al., 2008; Nolan, et al., “Role of interleukin-4 in regulation of age-related inflammatory changes in the hippocampus,” J. Biol. Chem., 2005; 280:9354-9362; Maher, et al., “Downregulation of IL-4-induced signalling in hippocampus contributes to deficits in LTP in the aged rat,” Neurobiol. Aging, 2005, 26:717-728), suggesting an increased classical activation phenotype over the alternative phenotype with age. It is generally agreed that microglia activated by extracellularly deposited Aβ protect neurons by triggering anti-inflammatory/neurotrophic M2 activation and by clearing Aβ via phagocytosis. This is a potential avenue for new therapeutic targets. (See, He, et al., 2007; Yamamoto, et al., “Interferon- gamma and tumor necrosis factor-alpha regulate amyloid-beta plaque deposition and beta- secretase expression in Swedish mutant APP transgenic mice,” Am. J. Pathol., 2007, 170:680-692; Yamamoto, et al., “Cytokine-mediated inhibition of fibrillar amyloid-beta peptide degradation by human mononuclear phagocytes,” J. Immunol., 2008,181:3877-3886).

Mantovani et al. (Mantovani, et al., 2004) studied the effect of IL-4 as an important modulator of M2a microglial activation. It has been shown that gene delivery of IL-4 into APP+PS1 mice partially suppressed glial accumulation in the hippocampus, directly enhanced neurogenesis, restored impaired spatial learning, and also reduced Aβ deposition (Kiyota, et al., 2010).

Yamamoto et al. (Yamamoto, et al., 2007, 2008) examined macrophage-mediated AP degradation using pro- and anti-inflammatory cytokines in primary cultured human monocyte-derived macrophages (MDM) and microglia. These studies showed that anti-inflammatory and regulatory cytokines lead to an increase in M2a or M2c activation and enhanced Aβ clearance. Kiyota et al. (Kiyota et al., 2011) have shown sustained expression of IL-4 reduced astro/microgliosis, amyloid-β peptide (Aβ) oligomerization and deposition, and enhanced neurogenesis.

Several approaches have been proposed to modulate microglial activation as potential targets for AD treatment. (See, Thériault, et al., 2015; Cherry, et al., “Neuroinflammation and M2 microglia: the good, the bad, and the inflamed,” J. Neuroinflammation, 2014, 11:98; Mandrekar, et al., 2010; Vernum, et al., 2012). It has been suggested that use of anti-inflammatory drugs, like non-steroidal anti-inflammatory drugs (NSAIDs), to halt the progression of AD could be suppressing both pro-inflammatory and anti-inflammatory activation by endogenous molecules, inactivating the beneficial effect of M2 microglial functions and endogenous mechanisms of plaque clearance. (See, Wilcock, et al., 2012, Cherry, et al., 2014; Theeriault, et al., 2015).

Research has focused primarily on two areas: anti-inflammatory agents to temper toxic effect of pro-inflammatory cytokines; and converting microglia from this M1 state to an M2 state in which the toxic effects are reduced and their phagocytic activity toward Aβ is enhanced. It was suggested (McGreer, et al., 2012) that potential treatments should be administered early in the disease progression.

Strategies that modulate monocyte and microglial activity have been studied, especially those that can protect against microglia-mediated neurotoxicity (Zhao, et al., 2006; Heneka, et al., 2013; Therlaut, et al., 2015; Nau, et al., 2014). Overall, it is clear that more focused studies need to be performed to better establish how each inflammatory state can modulate the pathologies of AD. It is generally accepted that early activation of monocytes and microglia has potential to decelerate AD progression by modulating immune responses to increase the intrinsic phagocytic capacity of monocytes and microglia without triggering secretion of pro inflammatory cytokines that could worsen AD.

### SUMMARY

In certain embodiments, the invention encompasses methods of treating a neuron inflammation condition comprising administering a therapeutically effective amount to a patient in need thereof of at least one compound having the following formula:

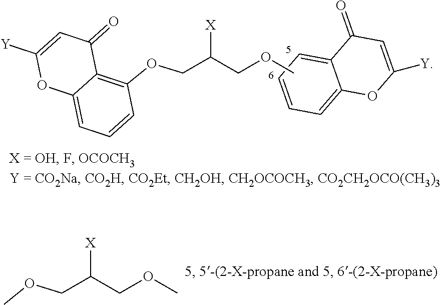

In other embodiments, the method uses the following compounds:

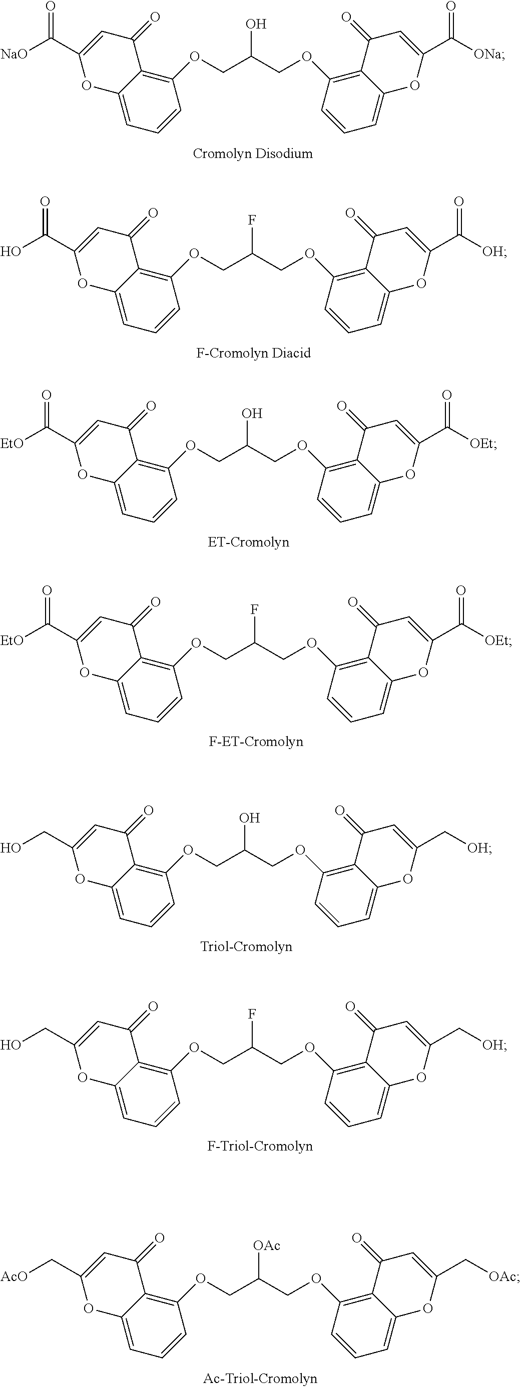

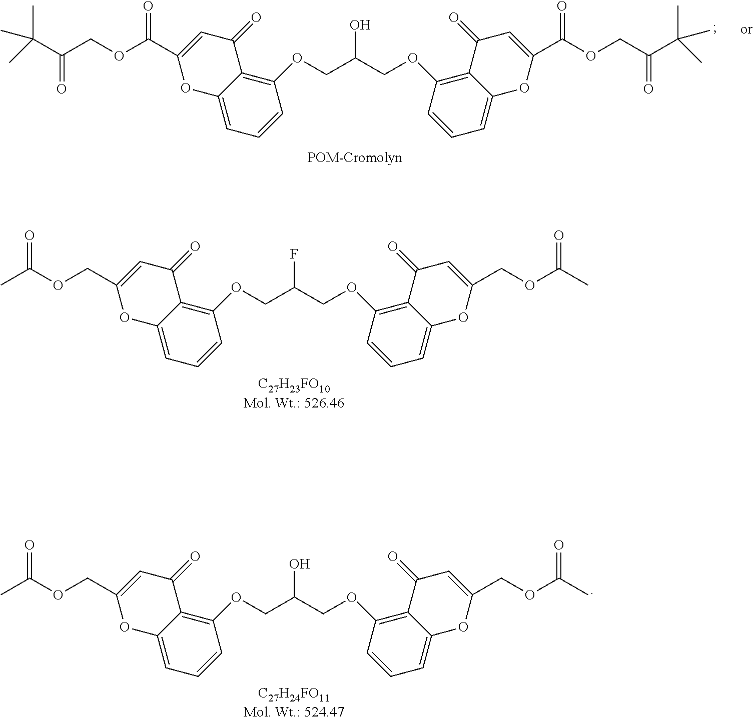

In yet other embodiments, the neuron inflammation condition is at least one of ALS, AD, ischemic stroke, or prion disease. In one embodiment, the compounds may be administered intraperitoneally (IP) and/or intravenously (IV). The compounds may be administered at a dose between about 1 mg and about 1000 mg per day. The method of administration may be transdermally or by inhalation.

In another embodiment, the method is a method of treating ALS further comprising co-administering CD4+; siRNA; miRNA that ameliorate ALS; glial morphology modifier; SOD1 control; Riluzole; or another M1; M2 conversion active drug that controls neuroinflammation.

In certain embodiments, the invention relates to any of the methods described herein, provided the compound is not cromolyn disodium. In certain embodiments, the invention relates to any of the methods described herein, provided the compound is not cromolyn disodium, F-cromolyn disodium, ET-cromolyn, or F-ET-cromolyn when the neuron inflammation condition is AD.

In certain embodiments, the invention relates to any one of the following compounds:

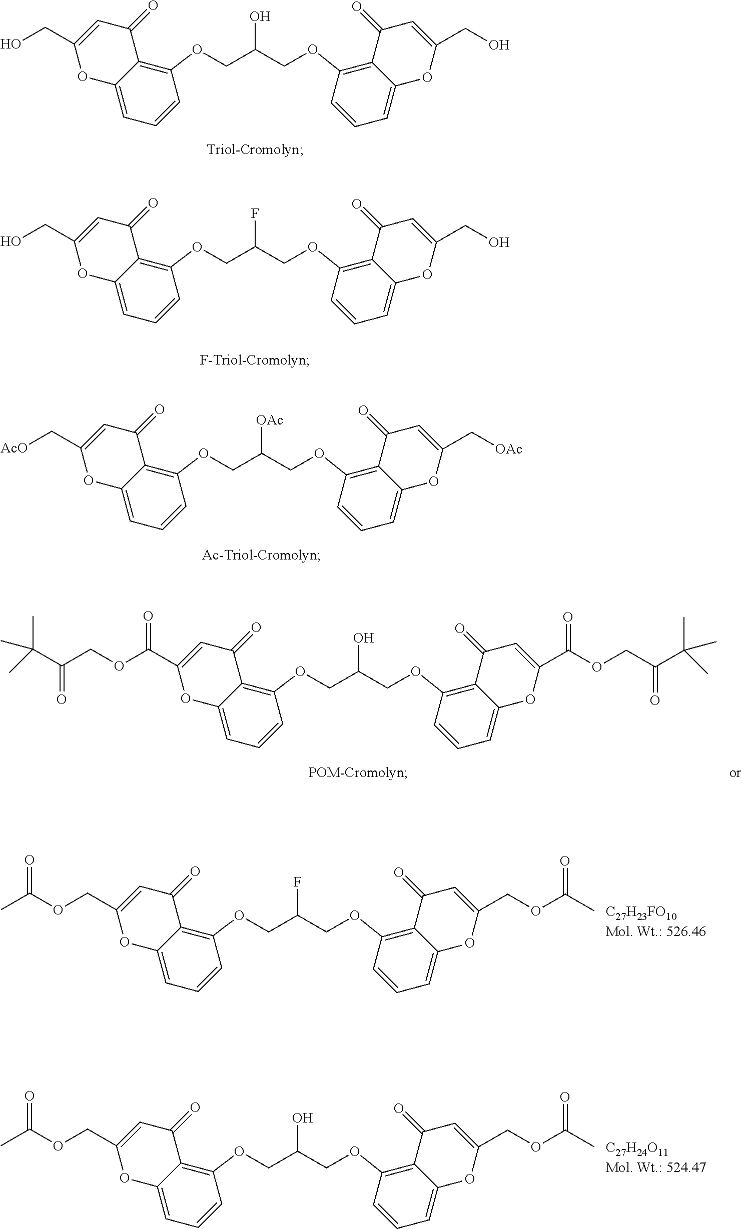

### BRIEF DESCRIPTION OF THE FIGURES

FIG. 1A illustrates the quantification of the plasmatic levels of Aβₓ₋₄₀ and Aβₓ₋₄₂ one week after treatment with PBS or escalating doses of Cromolyn Sodium (n=3-5 mice/group).

FIG. 1B illustrates representative images of localization of amyloid deposits (6E10) and microglia (Iba1 ) in mice treated with Cromolyn Sodium (3.1 5 mg/kg) or PBS daily for seven days. The bar figure illustrates the results from analyzing plaques for each animal. Scale bar=10 μm.

FIG. 1C illustrates the effect of Cromolyn Sodium on microglial Aβ uptake in vitro, where after the incubation, the concentrations of Aβₓ₋₄₀ (FIG. 1C left) Aβₓ₋₄₂ (FIG. 1C, right) in media were measured using Aβ ELISA.

FIG. 2 illustrates the plaques and the microglial cells surrounding those deposits in Tg-2576 mice of the study of Example 2. The figure shows representative pictures of amyloid deposits and Iba-1 positive microglia.

FIG. 3 illustrates the results of BV2 microglial cells treated with cromolyn, and with cromolyn and ibuprofen exhibit increased Aβ₄₂ uptake levels relative to BV2 microglia treated with the vehicle.

FIG. 4 illustrates the results of an Aβ aggregation inhibition assay using various compounds described herein.

FIG. 5 graphically illustrates that Cromolyn significantly affects the levels of brain TBS soluble Aβ and the ratios of Aβ (42:40).

FIG. 6A shows naïve BV2 microglial cells treated with DMSO (control) for 16 h. Afterwards, cells were incubated with fluorescently-labeled Aβ₄₂ and DMSO or cromolyn sodium for 2 hours. After incubation, cells were labeled with a plasma membrane dye (PM) and imaged.

FIG. 6B shows naïve BV2 microglial cells treated with DMSO (control) for 16 h. Afterwards, cells were incubated with fluorescently-labeled Aβ₄₂ and DMSO or cromolyn sodium for 2 hours.

FIG. 6C showns naïve BV2 microglial cells treated with cromolyn sodium (500 μM) for 16 hours. Afterwards, cells were incubated with fluorescently-labeled Aβ₄₂ and DMSO or cromolyn sodium for 2 hours. After incubation, cells were labeled with a plasma membrane dye (PM) and imaged.

FIG. 6D showns naïve BV2 microglial cells treated with cromolyn sodium (500 μM) for 16 hours. Afterwards, cells were incubated with fluorescently-labeled Aβ₄₂ and DMSO or cromolyn sodium for 2 hours.

FIG. 7A graphically illustrates that cromolyn sodium promotes microglial Aβ₄₂ uptake. BV2 microglial cells were treated with DMSO or different concentrations of cromolyn sodium for 16 hours. Then, cells were incubated with soluble untagged Aβ₄₂ and DMSO or cromolyn sodium for 2 hours, and collected for ELISA analysis. Both naïve BV2 and BV2-CD33^{WT} microglial cells treated with cromolyn sodium exhibited increased Aβ₄₂ uptake levels in comparison to cells treated with the vehicle (DMSO).

FIG. 7B graphically illustrates that cromolyn sodium promotes microglial Aβ₄₂ uptake. BV2 cells stably expressing CD33 (BV2-CD33^{WT} ) were treated with DMSO or different concentrations of cromolyn sodium for 16 hours. Then, cells were incubated with soluble untagged Aβ₄₂ and DMSO or cromolyn sodium for 2 hours, and collected for ELISA analysis. Both naïBV2 and BV2-CD33^{WT} microglial cells treated with cromolyn sodium exhibited increased Aβ₄₂ uptake levels in comparison to cells treated with the vehicle (DMSO).

FIG. 8 graphically illustrates that compound C8 displays toxicity when tested at 100 μM or higher concentration in LDH assay. Naïve BV2 microglial cells were treated with DMSO or cromolyn derivatives for 3 hours at different concentrations. C1, C2, C5, C6, C7 and C8 were tested at 10, 50, 100 and 150 μM, while C3 and C4 were assessed at 5, 25, 50 and 75 μM due to solubility limit in DMSO. Afterwards, cells were incubated with soluble untagged Aβ₄₂ peptide and DMSO or cromolyn derivatives for 2 hours. At the end of the treatment, cell media was collected and compound toxicity was assessed with the lactate dehydrogenase (LDH) assay. BV2 microglial cells treated with the cromolyn derivative C8 exhibited increased toxicity at 100 and 150 RM in comparison to cells treated with the vehicle (DMSO).

FIG. 9 graphically illustrates that compound C4 promotes Aβ₄₂ uptake in naïBV2 microglial cells. BV2 cells were treated with DMSO (vehicle) or cromolyn derivatives at different concentrations ranging from 5 to 150 μM for 3 hours. Then, cells were incubated with soluble untagged Aβ₄₂ and DMSO or cromolyn derivatives for additional 2 hours and collected for ELISA analysis. BV2 microglial cells treated with the cromolyn derivative C4 at 75 μM exhibited significantly increased Aβ₄₂ uptake levels in comparison to cells treated with the vehicle.

FIG. 10 graphically illustrates that compound C4 promotes Aβ₄₂ uptake in microglial BV2-CD33^{WT} cells. Microglial cells stably expressing CD33^{WT} were treated with DMSO as control or cromolyn derivatives (C1, C3-8) at different concentrations for 3 hours. Afterwards, cells were incubated with DMSO or cromolyn derivatives in the presence of the Aβ₄₂ peptide for additional 2 hours. Cell lysates were analyzed for intracellular levels of Aβ₄₂ using an Aβ₄₂-specific ELISA kit. Treatment with the cromolyn derivative C4 at 75 μM led to increased uptake of Aβ₄₂ in BV2-CD33^{WT} cells in comparison to DMSO treatment and displayed a dose-dependent effect at 50 μM.

FIG. 11 graphically illustrates that compound C4 promotes Aβ₄₂ uptake in BV2-CD33^{WT} cells. BV2-CD33^{WT} cells were treated with DMSO (vehicle) or cromolyn derivatives (C1, C2, and C4-7) at different concentrations for 3 hours. Afterwards, cells were treated with DMSO or cromolyn derivatives and soluble Aβ₄₂ peptide for 2 hours. Cell lysates were analyzed using Aβ₄₂-specific ELISA kit and intracellular Aβ₄₂ levels were quantified. The cromolyn derivative C4 effectively induced Aβ42 uptake at 50 and 75 μM in BV2-CD33^{WT} cells in comparison to cells treated with DMSO.

### DETAILED DESCRIPTION

Ischemic stroke, Alzheimer's Disease (AD), Amyotrophic Lateral Sclerosis (ALS or Lou Gehrig's disease), Prion and other neurodegenerative disorders are associated with microglial activation and mast cell migration, as well as with monocytes and other cell types that produce a barrage of toxic cytokines and debris that enhance inflammation. In certain embodiments, the invention encompasses anti-inflammatory compounds to reduce the toxic effect of pro-inflammatory cytokines by converting microglia from a pro-inflammatory M1 state to an M2 state in which the toxic effects are reduced and their phagocytic activity toward amyloidosis, tauopathies and other cytotoxic events is enhanced. In certain embodiments, the invention also encompasses the use of the compounds to affect therapy early in the disease process.

Many drugs used as anti-inflammatory agents showed no efficacy in the conversion of microglia from M1 to M2, nor do they enhance the modulation of microglia from M1 to M2. To the best of applicant's knowledge, the compounds described herein are the only effective, non-cytokine (e.g. IL-10) compounds exhibiting M1-to-M2 activity. Thus, in certain embodiments, the invention encompasses the compounds and the methods of treating neuron inflammation conditions by administration of a therapeutic effective amount of at least one of the compounds.

In certain embodiments, compounds of the invention include those having the following formula and their analogs and isomers:

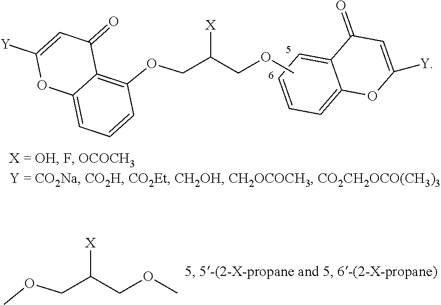

In addition, X may include, but is not limited to, halides, and OCO(C₁-C₈ alkyls). Alkyl groups include, but are not limited to, methyl, ethyl, propyl, isopropyl, butyl, isobutyl, tert-butyl, and pentyl. Halides include fluoro, chloro, bromo, and iodo. Y may include, but is not limited to, —CH₂OH, —CH₂OAc, or —CH₂OMe. Preferably, the compounds of the invention include those compounds attached at the 5 position.

Specific compounds with the scope of the invention include:

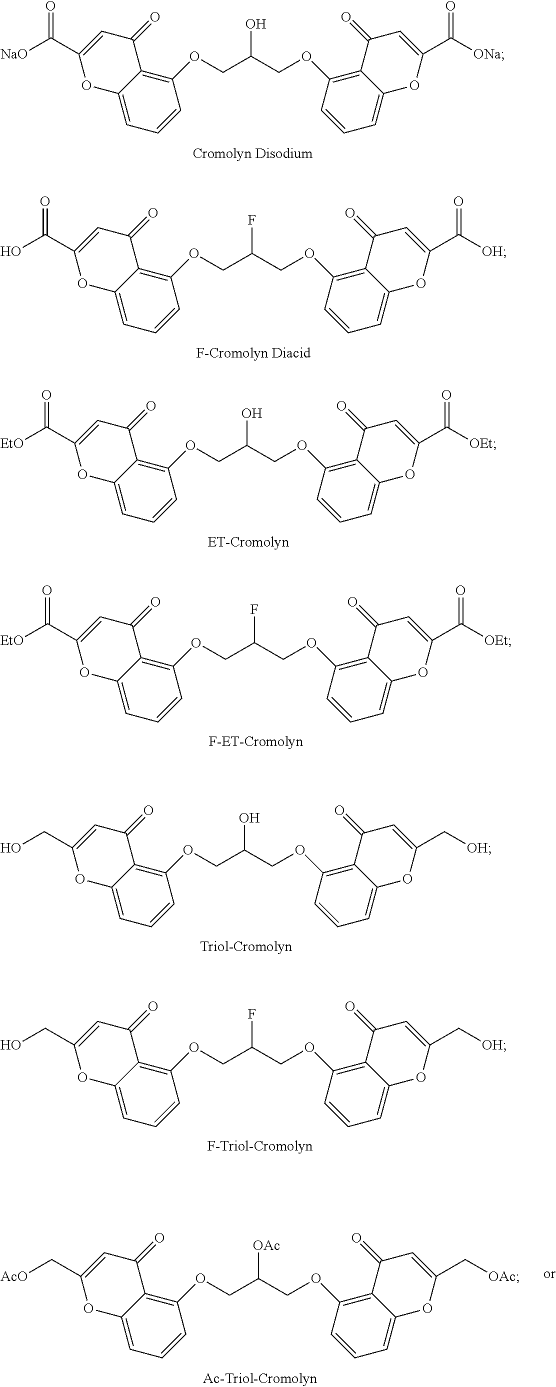

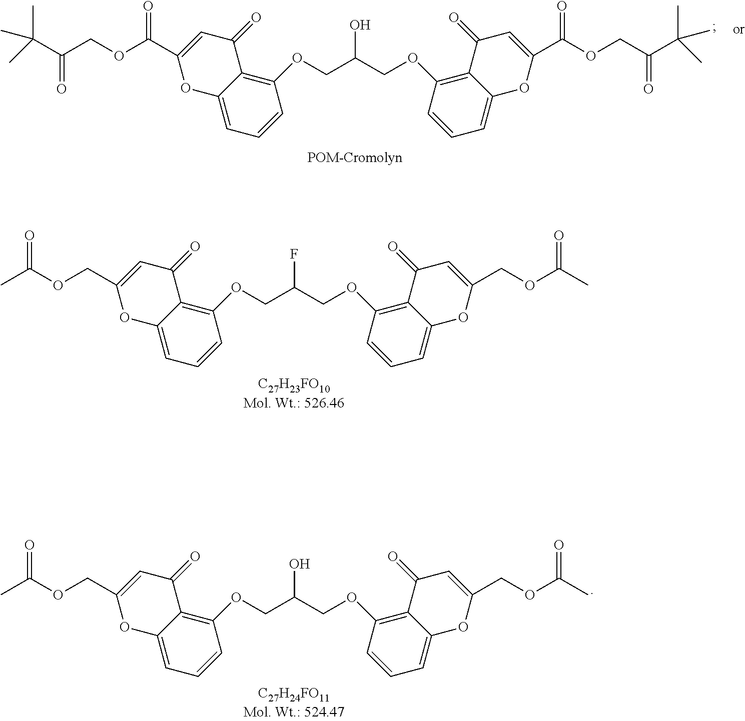

In certain embodiments, compounds also include 5-[3-(2-carboxy-4-oxochromen-5-yl)oxy-2-hydroxypropoxy]-4-oxochromene-2-carboxylic acid derivatives and isomeric forms.

In certain embodiments, the invention encompasses methods of treating a variety of neuron inflammation conditions. Neuron inflammation conditions include, but are not limited to, diseases such as ALS, autism spectrum disorder (ASD), ischemic stroke, and prion disease. In certain embodiments, the compounds may be used to treat ALS including, but not limited to, slowing down or halting the progression of the disease. In certain embodiments, the compounds may be administered in combination with other anti-inflammatory agents to control the spread of the progressive and fatal effect of ALS.

In certain embodiments, the invention encompasses a combination treatment for ALS of M1, M2 conversion active drugs that control neuroinflammation, such as the drugs in the above formulas, with other immune targeting therapies such as CD4+, siRNA, miRNA that ameliorates ALS, glial morphology modifiers, SOD1 controls, or Riluzole, the only approved drug for ALS.

In other embodiments, the compounds will slow down or halt neuron damage for neurons located in the brain stem and/or the spinal cord, neurons, or motor neurons that affect voluntary body muscles.

In certain embodiments, the compounds may be administered using known methods for the administration of drugs, for example, IP, IV, transdermally, by inhalation. In certain embodiments, the invention relates to methods of treating or slowing down the aggressive progression of a neurological disease, such as AD, Ischemic Stroke, ALS, or Prion, and the compound is administered by infusion or intraperitoneal administration.

In certain embodiments, the invention also provides pharmaceutical compositions comprising one or more compounds described herein in association with a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, auto-injector devices or suppositories; for oral, parenteral, intranasal, sublingual or rectal administration, or for administration by inhalation or insufflation. It is also envisioned that the compounds may be incorporated into transdermal patches designed to deliver the appropriate amount of the drug in a continuous fashion.

For preparing solid compositions such as powders and tablets, the principal active ingredient is mixed with a pharmaceutically acceptable carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture. When referring to these preformulation compositions as homogeneous, it is meant that the active ingredient is dispersed evenly throughout the composition so that the composition may be easily subdivided into equally effective unit dosage forms.

In some embodiments, a dry powder composition is micronized for inhalation to the lungs. See for example, U.S. Patent Application publication 2016/0263257, expressly incorporated herein by reference in its entirety, and in particular regarding the dry powder cromolyn formulations described therein. In other embodiments, the dry powder composition further comprises at least one excipient. In certain embodiments, the at least one excipient comprises Lactose monohydrate and/or Magnesium stearate.

In certain embodiments, the compounds may be administered in doses that treat the particular indication. In particular, the dose is specifically tailored to lead to blood, brain, and CSF concentrations that allow the drugs to act as M1-to-M2 modifiers. Such doses may include from about 1 mg to about 1000 mg per day.

The dosage of the active agents will generally be dependent upon a number of factors, including the pharmacodynamic characteristics of the compound, mode and route of administration of the compound, the health of the patient being treated, the extent of treatment desired, the nature and kind of concurrent therapy, if any, and the frequency of treatment and the nature of the effect desired. In general, dosage ranges of the compound often range from about 0.001 to about 250 mg/kg body weight per day. For a normal adult having a body weight of about 70 kg, a dosage may range from about 0.1 to about 25 mg/kg body weight. However, some variability in this general dosage range may be required depending on the age and weight of the subject being treated, the intended route of administration, the particular agent being administered, and the like. Importantly, the determination of dosage ranges and optimal dosages for a particular mammal is also well within the ability of one of ordinary skill in the art having the benefit of the instant disclosure.

Dosages for compounds may be as low as 5 ng/d. In certain embodiments, about 10 ng/day, about 15 ng/day, about 20 ng/day, about 25 ng/day, about 30 ng/day, about 35 ng/day, about 40 ng/day, about 45 ng/day, about 50 ng/day, about 60 ng/day, about 70 ng/day, about 80 ng/day, about 90 ng/day, about 100 ng/day, about 200 ng/day, about 300 ng/day, about 400 ng/day, about 500 ng/day, about 600 ng/day, about 700 ng/day, about 800 ng/day, about 900 ng/day, about 1μg/day, about 2 μg/day, about 3 μg/day, about 4 μg/day, about 5 μg/day, about 10 μg/day, about 15 μg/day, about 20 μg/day, about 30 mg/day, about 40 μg/day, about 50 μg/day, about 60 μg/day, about 70 μg/day, about 80 mg/day, about 90 μg/day, about 100 μg/day, about 200 μg/day, about 300 μg/day, about 400 μg/day about 500 μg/day, about 600 μg/day, about 700 μg/day, about 800 μg/day, about 900 μg/day, about 1 mg/day, about 2 mg/day, about 3 mg/day, about 4 mg/day, about 5 mg/day, about 10 mg/day, about 15 mg/day, about 20 mg/day, about 30 mg/day, about 40 mg/day or about 50 mg/day of the compound is administered.

Dosage ranges for active agents may be from 5 ng/d to 100 mg/day. In certain embodiments, dosage ranges for active agents may be from about 5 ng/day to about 10 ng/day, about 15 ng/day, about 20 ng/day, about 25 ng/day, about 30 ng/day, about 35 ng/day, about 40 ng/day, about 45 ng/day, about 50 ng/day, about 60 ng/day, about 70 ng/day, about 80 ng/day, about 90 ng/day, about 100 ng/day, about 200 ng/day, about 300 ng/day, about 400 ng/day, about 500 ng/day, about 600 ng/day, about 700 ng/day, about 800 ng/day, or about 900 ng/day. In certain embodiments, dosage ranges for compounds may be from about 1 μg/day to about 2 μg/day, about 3 μg/day, about 4 μg/day, about 5μg/day, about 10 μg/day, about 15 μg/day, about 20 μg/day, about 30 μg/day, about 40 μg/day, about 50 μg/day, about 60 μg/day, about 70 μg/day, about 80 μg/day, about 90 μg/day, about 100 μg/day, about 200 μg/day, about 300 μg/day, about 400 μg/day about 500 μg/day, about 600 μg/day, about 700 μg/day, about 800 μg/day, or about 900 μg/day. In certain embodiments, dosage ranges for active agents may be from about 1 mg/day to about 2 mg/day, about 3 mg/day, about 4 mg/day, about 5 mg/day, about 10 mg/day, about 15 mg/day, about 20 mg/day, about 30 mg/day, about 40 mg/day, about 50 mg/day, about 60 mg/day, about 70 mg/day, about 80 mg/day, about 90 mg/day, about 100 mg/day, about 200 mg/day, about 300 mg/day, about 400 mg/day, about 500 mg/day, about 600 mg/day, about 700 mg/day, about 800 mg/day, or about 900 mg/day.

In certain embodiments, the compounds are administered in pM or nM concentrations. In certain embodiments, the compounds are administered in about 1 pM, about 2 pM, about 3 pM, about 4 pM, about 5 pM, about 6 pM, about 7 pM, about 8 pM, about 9 pM, about 10 pM, about 20 pM, about 30 pM, about 40 pM, about 50 pM, about 60 pM, about 70 pM, about 80 pM, about 90 pM, about 100 pM, about 200 pM, about 300 pM, about 400 pM, about 500 pM, about 600 pM, about 700 pM, about 800 pM, about 900 pM, about 1 nM, about 2 nM, about 3 nM, about 4 nM, about 5 nM, about 6 nM, about 7 nM, about 8 nM, about 9 nM, about 10 nM, about 20 nM, about 30 nM, about 40 nM, about 50 nM, about 60 nM, about 70 nM, about 80 nM, about 90 nM, about 100 nM, about 300 nM, about 400 nM, about 500 nM, about 600 nM, about 700 nM, about 800 nM, or about 900 nM, concentrations.

In certain embodiments, the dosage form is a solid dosage form, and the size of the compound in the dosage form is important. In certain embodiments, the compound is less than about 3 μm, less than about 2 μm, or less than about 1 μm in diameter. In certain embodiments, the active agent is about 0.1 μm to about 3.0 μm in diameter. In certain embodiments, the active agent is from about 0.5 82 m to about 1.5 μm in diameter. In certain embodiments, the active agent is about 0.2 μm, about 0.3 μm, about 0.4 μm, about 0.5 μm, about 0.6 μm, about 0.7 μm, about 0.8 μm, about 0.9 μm, about 1.0 μm, about 1.1 μm, about 1.2 μm, about 1.3 μm, about 1.4 μm, or about 1.5 μm in diameter.

For example, a formulation intended for oral administration to humans may contain from about 0.1 mg to about 5 g of the active agent (or compound) compounded with an appropriate and convenient carrier material varying from about 5% to about 95% of the total composition. Unit dosages will generally contain between about 0.5 mg to about 1500 mg of the active agent. The dosage may be about: 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg 25 mg, 26 mg, 27 mg, 28 mg, 29 mg, 30 mg, 31 mg, 32 mg, 33 mg, 34 mg 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 55 mg, 60 mg, 65, mg, 70 mg, 75 mg, 80 mg, 85 mg, 90 mg, 95 mg, 100 mg, 200 mg, 300 mg, 400 mg, 500 mg, 600 mg, 800 mg, or 100 mg, etc., up to about 1500 mg of the compound.

In certain embodiments, the invention relates to combination of two active agents. In certain embodiments, it may be advantageous for the pharmaceutical combination to be comprised of a relatively large amount of the first component compared to a second component. In certain instances, the ratio of the first active agent to the second active agent is about: 200:1, 190:1, 180:1, 170:1, 160:1, 150:1, 140:1, 130:1, 120:1, 110:1, 100:1, 90:1, 80:1, 70:1, 60:1, 50:1, 40:1, 30:1, 20:1, 15:1, 10:1, 9:1, 8:1, 7:1, 6:1, or 5:1. It further may be preferable to have a more equal distribution of pharmaceutical agents. In certain instances, the ratio of the first active agent to the second active agent is about: 4:1, 3:1, 2:1, 1:1, 1:2, 1:3, or 1:4. It may also be advantageous for the pharmaceutical combination to have a relatively large amount of the second component compared to the first component. In certain instances, the ratio of the second active agent to the first active agent is about 200:1, 190:1, 180:1, 170:1, 160:1, 150:1, 140:1, 130:1, 120:1, 110:1, 100:1, 90:1, 80:1, 70:1, 60:1, 50:1, 40:1, 30:1, 20:1, 15:1, 10:1, 9:1, 8:1, 7:1, 6:1, or 5:1. A composition comprising any of the above identified combinations of the first therapeutic agent and second therapeutic agent may be administered in divided doses about 1, 2, 3, 4, 5, 6, or more times per day or in a form that will provide a rate of release effective to attain the desired results. The dosage form may contain both the first and second active agents. The dosage form may be administered one time per day if it contains both the first and second active agents.

For example, a formulation intended for oral administration to humans may contain from about 0.1 mg to about 5 g of the first therapeutic agent and about 0.1 to about 5 g of the second therapeutic agent, both of which are compounded with an appropriate and convenient about of carrier material varying from about 5% to about 95% of the total composition. Unit dosages will generally contain between about 0.5 mg to about 1500 mg of the first therapeutic agent and 0.5 mg to 1500 mg of the second therapeutic agent. The dosage may be about: 25 mg, 50 mg, 100 mg, 200 mg, 300 mg, 400 mg, 500 mg, 600 mg, 800 mg, or 100 mg, etc., up to about 1500 mg of the first therapeutic agent. The dosage may be about: 25 mg, 50 mg, 100 mg, 200 mg, 300 mg, 400 mg, 500 mg, 600 mg, 800 mg, or 100 mg, etc., up to about 1500 mg of the second therapeutic agent.

In certain embodiments, the inventions relates to a method of treating a Alzheimer's disease comprising administering by inhalation a micronized, dry powder comprising about 1 mg to 100 mg of Cromolyn Disodium per day to a patient in need thereof.

### EXAMPLES

### Example 1

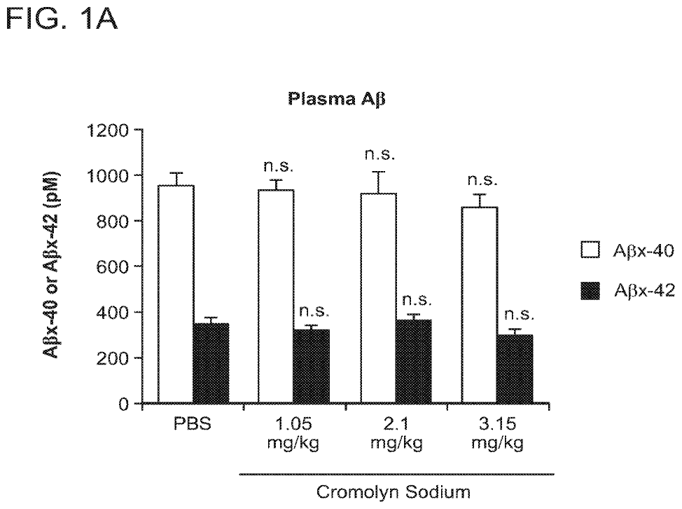

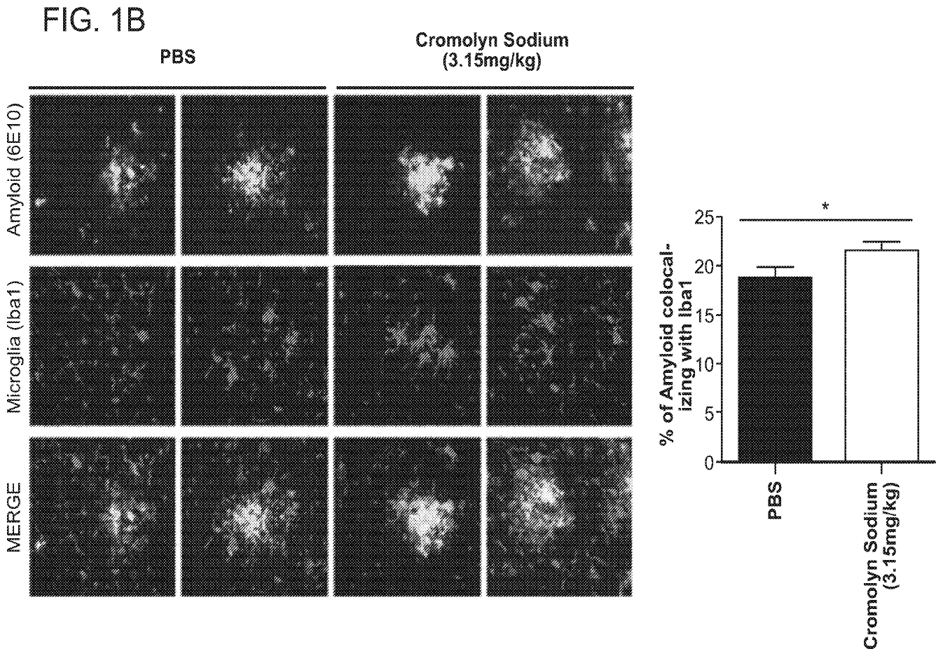

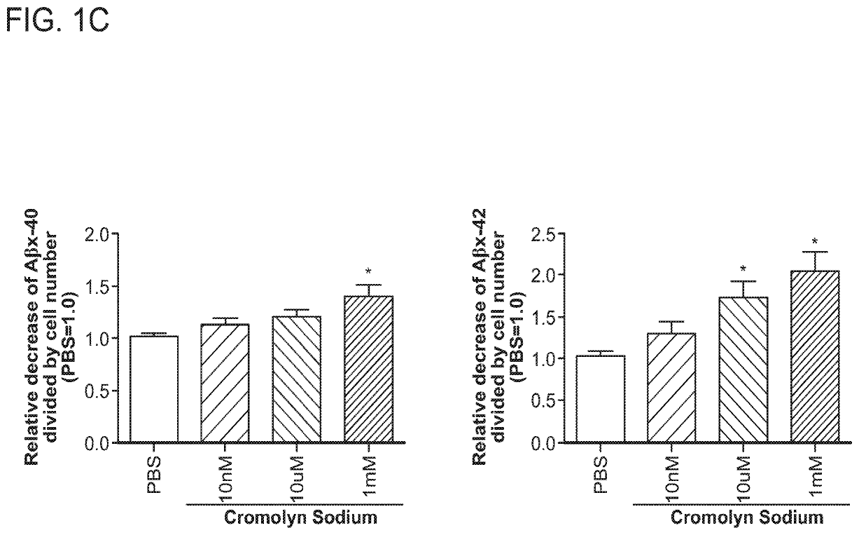

Our studies in PS1/PSS animal model showed that Cromolyn sodium impacted the interaction of microglial cells with amyloid deposits and eventually affected Aβ clearance by microglia. We first performed a double immunostaining between Aβ and the microglial marker Iba1 in brain sections of mice treated with PBS or the highest dose of Cromolyn sodium (3.15 mg/kg). A systematic analysis of the overlap between both stainings revealed that animals that received Cromolyn Sodium showed a higher percentage of Iba1 immunoreactivity overlapping with amyloid (FIG. 1B), which may indicate a modest increased recruitment of microglia around plaques induced by the compound.

To go further in our understanding of these mechanisms, and considering that evaluating change in microglial function is challenging in vivo, we used an additional in vitro system of Aβ microglial uptake. Synthetic Aβ₄₀ and Aβ₄₂ peptides were applied to microglia in culture in the presence or absence of Cromolyn Sodium.

After 16 hours of incubation, we observed a dose dependent decrease of Aβ₄₀ and Aβ₄₂ levels in presence of Cromolyn Sodium, indicating that the impact of Cromolyn Sodium on Aβ aggregation mechanisms may promote Aβ clearance by microglial uptake (FIG. 1C). The combination of those in vivo and in vitro results may suggest that, in addition to inhibiting Aβ fibrillization, Cromolyn Sodium affected microglial activation and Aβ clearance.

Cromolyn Sodium does not affect the levels of Aβ in the plasma but promotes microglial Aβ clearance. FIG. 1A illustrates the quantification of the plasmatic levels of Aβₓ₋₄₀ and Aβₓ₋₄₂ one week after treatment with PBS or escalating doses of Cromolyn Sodium (n=3-5 mice/group). FIG. 1B illustrates representative images of localization of amyloid deposits (6E10) and microglia (Iba1) in mice treated with Cromolyn Sodium (3.15 mg/kg) or PBS daily for seven days. The percentage of amyloid occupied by Iba1 positive processes was calculated for each deposit and showed an increased overlap between Aβ and Iba1 after treatment with Cromolyn Sodium (n=3 mice for PBS and n=5 mice for Cromolyn Sodium). Between 20 to 20 plaques were evaluated for each animal). Scale bar=10 μm. FIG. 1C illustrates the effect of Cromolyn Sodium on microglial Aβ uptake in vitro. Microglial cells were cultured and incubated with 50 nM of synthetic Aβ₄₀ or Aβ₄₂ and 0, 10 nM, 10 μM or 1 mM of Cromolyn Sodium for 16 hours. After the incubation, the concentrations of Aβₓ₋₄₀ (FIG. 1C left) Aβₓ₋₄₂ (FIG. 1C, right) in media were measured using Aβ ELISA and normalized with microglia cell number and according to the PBS control condition. (n=3 experiments; *,P<0.05, **P<0.01)

### Example 2

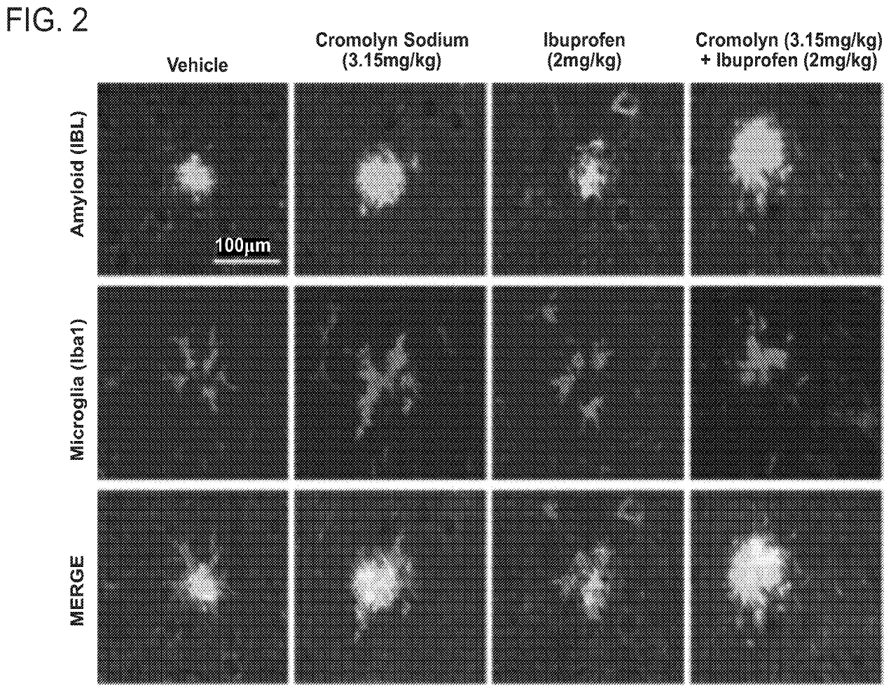

In other animal studies of microglia activation and M1, M2 conversion showed that cromolyn is the only of many drugs tested that effected this change and exhibited phagocytic activity. FIG. 2 illustrates representative plaques of all the plaques and the microglial cells surrounding those deposits in Tg-2576 mice of the study. An image analysis looking at the percentage of Iba-1 positive processes colocalizing with the amyloid staining versus the total amount of Iba-1 signal surrounding the plaque demonstrated that there was more Iba-1/Amyloid colocalization when the mice were treated with Cromolyn Sodium as opposed to any other groups. This result correlates with our results in Example 1 and our in vitro data.

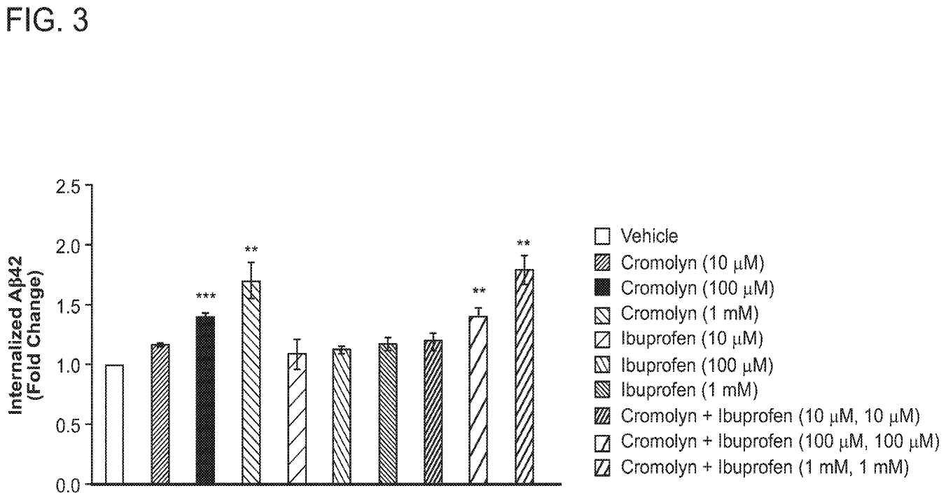

Cromolyn, but not ibuprofen promotes microglial Aβ₄₂ uptake, their combination improved uptake over either ibuprofen or cromolyn alone. BV2 microglial cell cultures were treated with cromolyn and/or ibuprofen (10 μM, 100 μM, 1 mM) for 16 hours. Afterwards, cells were incubated with soluble Aβ₄₂ and the compounds for 3 hours. After incubation, cells were collected for ELISA analysis. BV2 microglial cells treated with cromolyn (100 μM, 1 mM), and with cromolyn and ibuprofen (100 μM, 1 mM for each compound) exhibit increased Aβ₄₂ uptake levels relative to BV2 microglia treated with the vehicle. Results were derived from three independent experiments; **p <0.01, ***p <0.001, one-way ANOVA, Tukey's test). Data are represented as mean±SEM. FIG. 3 graphically illustrates the results of BV2 microglial cells treated with cromolyn, and with cromolyn and ibuprofen exhibit increased Aβ₄₂ uptake levels relative to BV2 microglia treated with the vehicle.

### Example 3: Compound Synthesis

### 5,5′-[(2-Hydroxy-1,3-propanediyl)bis(oxy)]bis[4-oxo-4H-1-benzopyran-2-carboxylic acid diethyl ester

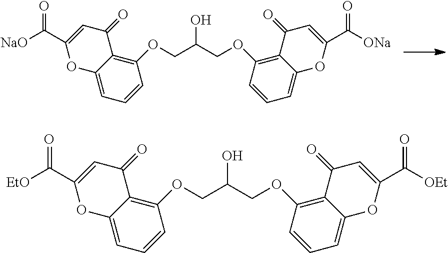

A suspension of cromolyn sodium salt (1.0 g, 2 mmol) in EtOH (100 mL) and con. HCl (1 mL) was heated in a sealed reactor tube for 24 h at 100° C. The white solid was dissolved to give a clear colorless solution while hot. It was allowed to cool to room temperature and NaHCO₃ (1.0 g) was added. After stirring for 30 min at 25° C., solvent was removed by roto-evaporation. Chromatography on silica gel of the crude material using 5:95 methanol/methylene chloride yielded the diethyl ester (0.8 g, 76% yield); mp 154-156° C.; ¹H NMR (CDCl₃, 300 MHz) δ 1.42 (t, 3H, J=7.1 Hz, CH₃), 2.73 (br s, 1H, OH), 4.44 (q, 4H, J=7.1 Hz, 2OCH₂CH₃), 4.32-4.59 (m, 5H, CHOH, 2OCH₂), 6.80 (s, 2H, 2 vinyl-H), 6.99 (d, 2H, J=8.24 Hz, 2Aro-H), 7.12 (d, 2H, J=8.24 Hz, 2Aro-H), 7.17 (d, 2H, J=8.24 Hz, 2Aro-H), 7.71 (t, 2H, J=8.24 2Aro-H).

### 5,5′-[(2-Fluoro-1,3-propanediyl)bis(oxy)]bis[4-oxo-4H-1-benzopyran-2-carboxylic acid diethyl ester

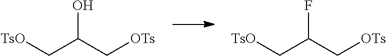 [SMILES: Cc1ccc(S(=O)(=O)OCC(O)COS(=O)(=O)c2ccc(C)cc2)cc1 ; Cc1ccc(S(=O)(=O)OCC(F)COS(=O)(=O)c2ccc(C)cc2)cc1]

### 3-Bis(4-methylbezenesulfonate)-2-fluoropropanediol

A solution of 1,3-bis(4-methylbezenesulfonate propanetriol (2.7 g, 6.78 mmol) in methylene chloride (20 mL) at 0-5° C. was treated with DAST (2.18 g, 13.6 mmol). The mixture was stirred at 0-5° C. for 30 then allowed to warm to 25° C. and stirred for 16 hr. The mixture was poured into a sat'd sodium bicarbonate solution (30 mL) and layers separated. The methylene chloride layer dried (sodium sulfate). After solvent removal, the crude material was chromatographed on silica gel (methylene chloride) to yield 0.82 g (30%) of a solid; mp 99-102° C.; ¹H NMR (CDCl₃), δ 2.5 (s, 6H, CH₃), 4.15 (dd, 4H, J=12.3, 4.6 Hz,CH₂, 4.8 (dq, 1H, J=47, 4.6, CHF), 7.45 (d, 4H, J=8.1 Hz, Aro-H), 7.75 (d, 4H, J=8.4 Hz, Aro-H).

### 5,5′-(2-fluoropropane-1,3-diyl)bis(oxy)bis(4-oxo-4H-chromene-2-carboxylic acid)

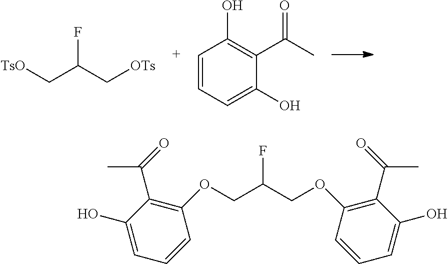 [SMILES: Cc1ccc(S(=O)(=O)OCC(F)COS(=O)(=O)c2ccc(C)cc2)cc1 ; CC(=O)c1c(O)cccc1O ; CC(=O)c1c(O)cccc1OCC(F)COc1cccc(O)c1C(C)=O]

### 1,3-Bis(2-acetyl-3-hydroxyphenoxy)-2-fluoropropane

A mixture of 3-bis(4-methylbezenesulfonate)-2-fluoropropanediol (1.0, 2.5 mmol), 2,6-dihydroxyacetophenone (0.76 g, 5.0 mmol) and potassium carbonate (0.69 g) in acetonitrile (40 mL) was heated under reflux for 16 hr. The mixture was filtered and the filtrate was evaporated. The crude material was chromatographed on silica gel (acetonitrile/methylene chloride 5:95) to yield 0.57 g (40%) of product; mp 162-165° C.; ¹H NMR (d6-DMSO), δ 2.5 (s, 6H, 2CH₃), 4.38 (m, 4H, 2CH₂), 5.22 (br d 1H, J=49 Hz, CHF), 6.45 (m, 4H, 4Aro-H), 7.28 (t, 2H, J=4.55 Hz, 2Aro-H).

### 5,5′-[(2-Fluoro-1,3-propanediyl)bis(oxy)]bis[4-oxo-4H-1-benzopyran-2-carboxylic acid diethyl ester

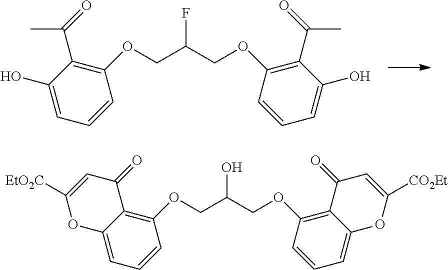 [SMILES: CC(=O)c1c(O)cccc1OCC(F)COc1cccc(O)c1C(C)=O ; CCOC(=O)c1cc(=O)c2c(OCC(O)COc3cccc4oc(C(=O)OCC)cc(=O)c34)cccc2o1]

A mixture of 1,3-bis(2-acety-3-hydroxyphenoxy)-2-fluoropropane (200 mg, 0.52 mmol) and ethyl oxalate (2 mL) was added to a solution of sodium ethoxide (87 mg Na) in ethanol (10 mL) and benzene (10 mL). The mixture was heated at reflux for 16 hr, cooled and diluted with ether (50 mL). The precipitated sodium salt was filtered, washed with ether and dried. It was then dissolved in water and acidified with 10% HCl to obtain a sticky solid. The solid was refluxed in ethanol (20 mL) with a catalytic amount of 36% HCL for 1 hr. The mixture was poured into 50 mL of water and extracted twice with methylene chloride (50 mL). The extracts were combined and dried. After solvent removal, the crude material was chromatographed on silica gel (acetonitrile/methylene chloride 10:90) to yield 0.12 g (45%) of product; mp 166-170° C.; ¹H NMR (CDCl₃), δ 1.42 (t, 6H, J=7.14 Hz, 2CH₃), 4.58 (q, 4H, J=7.14 Hz 2CH₂), 4.65 (m, 4H, 2CH₂), 5.35 (dq, 1H, J=46 Hz, J=4.4 HZ, CHF), 6.90 (s, 2H, vinyl-H), 6.95 (d, 2H, J=8.24 Hz, 2Aro-H), 7.13(d, 2H, J=8.24 Hz, 2Aro-H),7.17 (d, 2H, J=8.24 Hz, 2Aro-H) 7.6 (t, 2H, J=8.24 2Aro-H).

### 5,5′-[(2-Fluoro-1,3-propanediyl)bis(oxy)]bis[4-oxo-4H-1-benzopyran-2-carboxylic acid

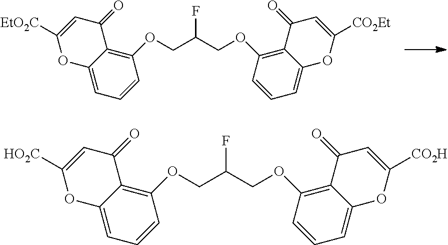

A suspension of 5,5′-[(2-fluoro-1,3-propanediyl)bis(oxy)]bis[4-oxo-4H-1-benzopyran-2-carboxylic acid diethyl ester (100 mg, 0.19 mmol) in methanol (20 mL) and 1 M sodium hydroxide (2 mL) was heated at 80° C. for 1 hr. The solution was acidified with 10% HCl and volatiles were removed. A solution of methanol/methylene chloride (50:50) was added to the solid and the mixture was filtered. Evaporation afforded 76 mg (85%) of product; ¹H NMR (d6-DMSO), δ 4.65 (m, 4H, 2CH₂), 5.32 (br d, 1H, J=46 Hz, CHF), 6.80 (s, 2H, 2vinyl-H), 7.2 (d, 2H, J=8.24 Hz, 2Aro-H), 7.71 (t, 2H, J=8.24 2Aro-H).

### 5,5′-[(2-Hydroxy-1,3-propanediyl)bis(oxy)]bis[4-oxo-4H-1-benzopyran-2-ethanol

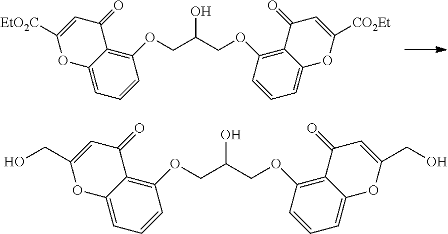

To a suspension of 5,5′-(2-hydroxytrimethylenedioxy)bis(4-oxochromene-2-carboxylic acid) diethyl ester (1.0 g, 1.86 mmol) in methanol (60 ml) and methylene chloride (40 mL) NaBH₄ (0.14 g, 3.72 mmol) was added in portions over a 1 h period. The mixture was stirred at 25° C. until it was clear (approx. 5 h) at which time the solution was quenched by dropwise addition of 1M HCl until acidic. Solvent was evaporated and the residue was extracted with methylene chloride. The combined organic extracts were washed with water and dried over anhydrous sodium sulfate. After evaporation, the residue was purified by column chromatography (5:95 methanol/methylene chloride) to yield 0.5 g (50%) of the triol; ¹H NMR (DMSO-d₆, 300 MHz) δ 2.73 (s, 3H, OH), 4.25-4.36 (m, 9H, 2OCH₂, CH—O), 6.13 (s, 2H, 2 vinyl H), 7.04 (d, 2H, J=8.4 Hz, aromatic H), 7.07 (d, 2H, J=8.4 Hz, aromatic H), 7.63 (t, 2H, J=8.2 Hz, aromatic H).

### 5,5′-[(2-Fluoro-1,3-propanediyl)bis(oxy)]bis[4-oxo-4H-1-benzopyran-2-ethanol

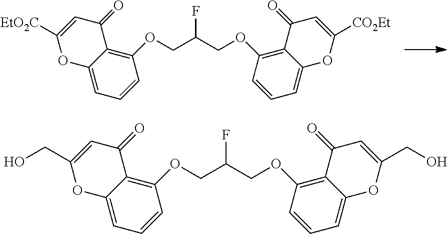

The above procedure for 5,5′-(2-hydroxytrimethylenedioxy)bis(4-oxochromene-2-ethanol) was used. ¹H NMR (DMSO-d₆, 300 MHz) δ 2.73 (s, 3H, OH), 4.25-4.36 (m, 8H, 2OCH₂, CH—O), 5.35 (br d, 1H, J=46 Hz, CHF), 6.13 (s, 2H, 2 vinyl H), 7.04 (d, 2H, J=8.4 Hz, aromatic H), 7.07 (d, 2H, J=8.4 Hz, aromatic H), 7.63 (t, 2H, J=8.2 Hz, aromatic H).

### 5,5′-[(2-Hydroxy-1,3-propanediyl)bis(oxy)]bis[4-oxo-4H-1-benzopyran-2-carboxylic acid bis[(2,2-dimethyl-1-oxopropoxy)methyl] ester

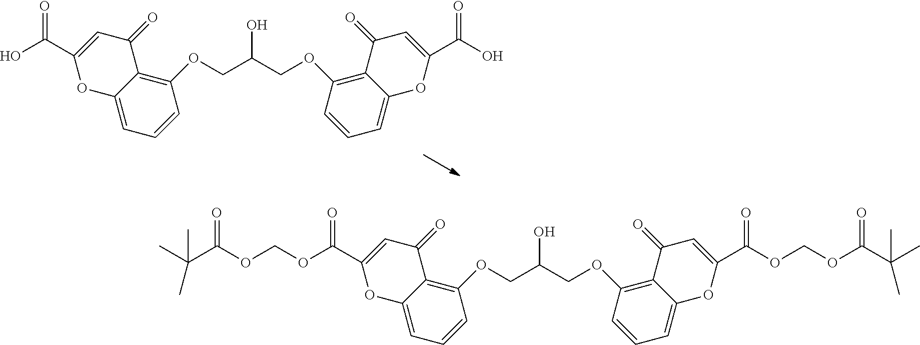 [SMILES: O=C(O)c1cc(=O)c2c(OCC(O)COc3cccc4oc(C(=O)O)cc(=O)c34)cccc2o1 ; CC(C)(C)C(=O)OCOC(=O)c1cc(=O)c2c(OCC(O)COc3cccc4oc(C(=O)OCOC(=O)C(C)(C)C)cc(=O)c34)cccc2o1]

To a stirred solution of cromolyn diacid (1.0 g, 2.7 mm) in 20 mL of DMF was added diisopropylamine (0.7mL) and 1.0 g (6.5 mmol) chloromethylpivalate. The reaction mixture was stirred at 60 ° C. for 4 hr, water was added and the mixture was extracted with separated, dried (MgSO₄) and the solvent removed in vacuo. The solvent was removed and the residue was chromatographed on silica 4% methanol in methylene chloride to give 1.2 g (65%) of the pivalate compound; mp 135-140° C.; H¹ NMR (CDCl), δ 1.24 (s, 18H, CH₃), 4.36 (m, 2 H, OCH₂), 4.49 (m, 1H, CHOH), 4.51 (m, 2H, OCH₂),), 6.00 (s, 4H, CH—O—CO), 6.98 (m, 4H, 2vinyl-H, 2Aro-H), 7.13 (d, 2H, J=8.24 Hz, 2Aro-H), 7.61 (t, 2H, J=8.24 2Aro-H).

### 5,5′-[(2-Fluoro-1,3-propanediyl)bis(oxy)]bis[4-oxo-4H-1-benzopyran-2-carboxylic acid bis[(2,2-dimethyl-1-oxopropoxy)methyl] ester

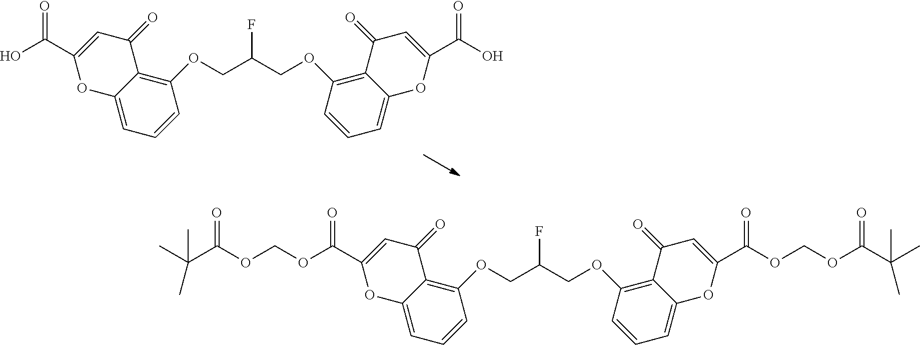 [SMILES: O=C(O)c1cc(=O)c2c(OCC(F)COc3cccc4oc(C(=O)O)cc(=O)c34)cccc2o1 ; CC(C)(C)C(=O)OCOC(=O)c1cc(=O)c2c(OCC(F)COc3cccc4oc(C(=O)OCOC(=O)C(C)(C)C)cc(=O)c34)cccc2o1]

To a stirred solution of 5,5′-[(2-fluoro-1,3-propanediyl)bis(oxy)]bis[4-oxo-4H-1-benzopyran-2-carboxylic acid (1.0 g, 2.1 mmol) in 20 mL of DMF was added iisopropylamine (0.7 mL) and 1.0 g (6.5 mmol) chloromethylpivalate. The reaction mixture was stirred at 60° C. for 4 hr, water was added and the mixture was extracted with separated, dried (MgSO₄) and the solvent removed in vacuo. The solvent was removed and the residue was chromatographed on silica using 2% methanol in methylene chloride to give 1.0 g (70%) of the pivalate compound; mp 130-133° C.; δ 1.21 (s, 18 H, CH₃), 4.36 (m, 4 m, 2OCH₂), 4.49 (br d, 1H, J=46 Hz, CHF), 6.00 (s, 4H, CH—O—CO), 6.98 (m, 4H, 2vinyl-H, 2Aro-H), 7.13 (d, 2H, J=8.24 Hz, 2Aro-H), 7.61 (t, 2H, J=8.24 2Aro-H).

### Triacetate of 5,5′-[(2-hydroxy-1,3-propanediyl)bis(oxy)]bis[4-oxo-4H-1-benzopyran-2-ethanol

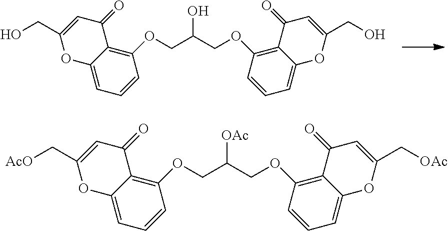 [SMILES: O=c1cc(CO)oc2cccc(OCC(O)COc3cccc4oc(CO)cc(=O)c34)c12 ; CC(=O)OCc1cc(=O)c2c(OCC(COc3cccc4oc(COC(C)=O)cc(=O)c34)OC(C)=O)cccc2o1]

Acetic anhydride (0.5 g, 4.6 mmol)) was slowly added to a mixture of 5,5′-[(2-hydroxy-1,3-propanediyl)bis(oxy)]bis[4-oxo-4H-1-benzopyran-2-ethanol (0.5 g, 1.14 mmol) in pyridine (20 mL) cooled to 0-5° C. The mixture was stirred for 3 hr at 0-5° C. and then allowed to warm to room temperature. TLC indicted the reaction was complete. Methylene chloride was added and the mixture was washed with 10% HCl until the aqueous phase was acidic. The methylene chloride layer was dried over anhydrous sodium sulfate and solvent was evaporated. Chromatography on silica using 3% methanol in methylene chloride gave 0.45 g (72%) of the triacetate compound; mp 122-125° C.; H¹ NMR (CDCl₃), 67 2.16 (s, 9H, CH₃), 4.58 (m, 2H, CH₂OH), 4.66 (m, 2H, CH₂OH), 4.94 (s, 4H, CH₂OH), 5.66 (m, 1H, CHOH), 6.15 (s, 2H, 2vinyl-H), 6.94 (d, 2H, 2Aro-H), 6.97 (d, 2H, J=8.24 Hz, 2Aro-H), 7.52 (t, 2H, J=8.24, 2Aro-H).

### Example 4: Aβ Aggregation Inhibition Assay

Experimental design. 3-month old Tg2576 mice were acclimatized for 2 months, and then randomly assigned to different treatment groups. They included the control group (n=10) with vehicle treatment, the cromolyn low dose group and cromolyn high dose group. The treatments were conducted through IP injection with PBS based on 0.1 mL/30g body weight, 3 times per week for 3 additional months. All mice were sacrificed at 8-month old. Tissues were harvested and processed for postmortem analysis.

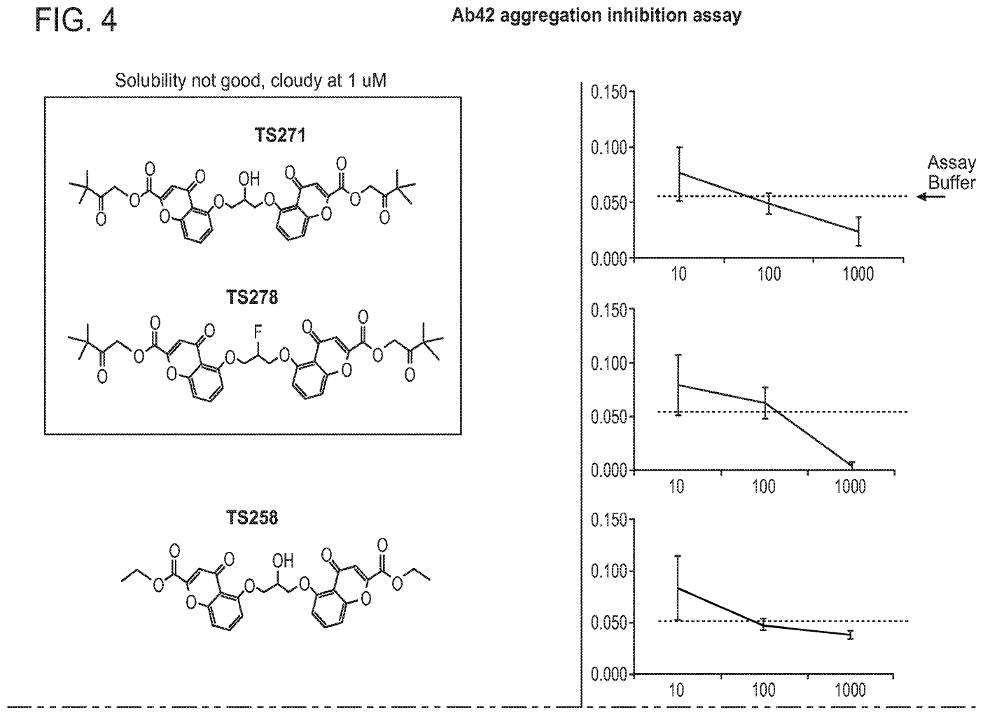

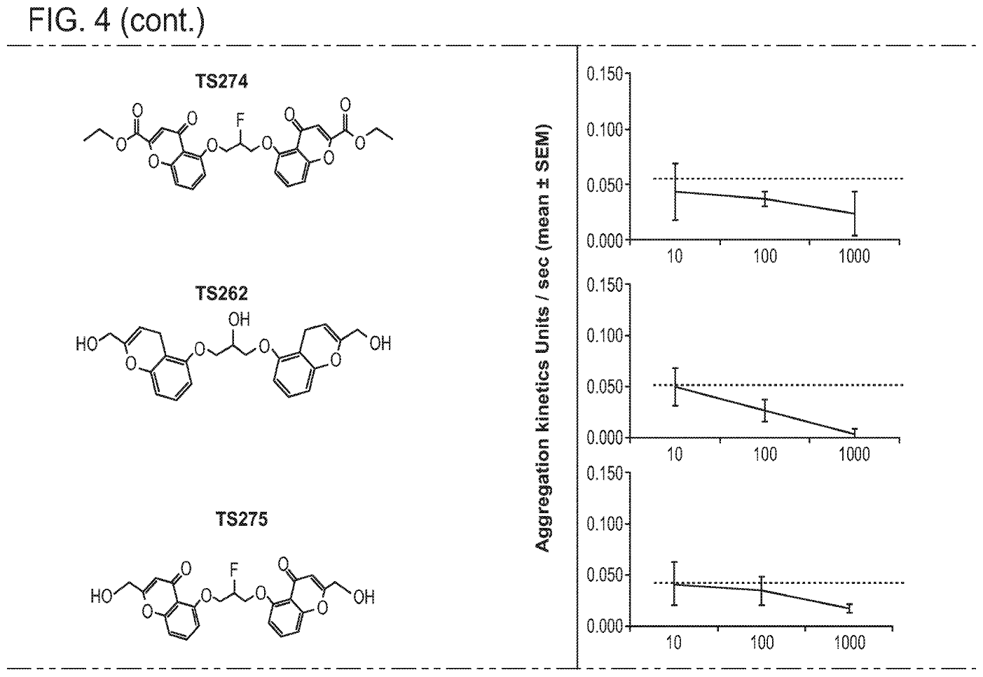

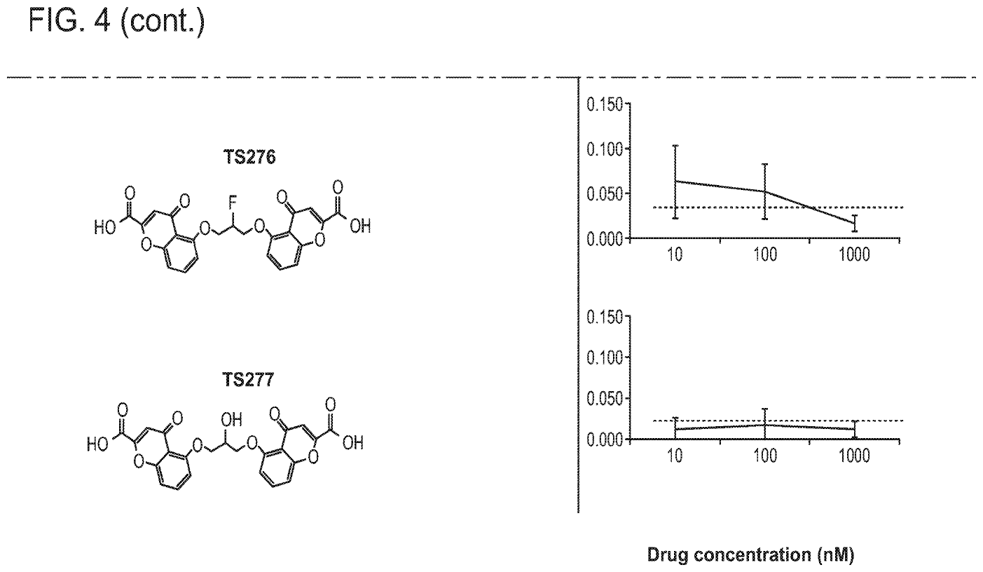

Synthetic Aaβ₄₂ in final 5 uM was incubated with 10, 100, 1,000 nM of test compounds for 1 hour. The aggregation was initiated with heparin at 0.5 mg/ml in final concentration. The assay buffer consisted of 125 mM NaCl, 2.5 mM KCl, mM MgCl₂, 1.25 mM Na₂H₂PO₄, 2 mM CaCl₂, 25 mM Glucose, and NaHCO₃ to adjust pH to 7.4. The assay buffer was used as a control. The aggregation was measured by intensity of Thioflavin T binding, which was detected by fluorescent excitation/emission at 450 nm/480 nm (Spectra Max M3 plate reader, Molecular Devices) in a kinetic mode. Aggregation was recorded as the kinetics was calculated as Vmax by the plate reader's software. The assay was performed in triplicate and expressed as standard mean ±SD. Blue dotted line indicate the assay buffer control. FIG. 4 illustrates the results of the assay.

### Example 5

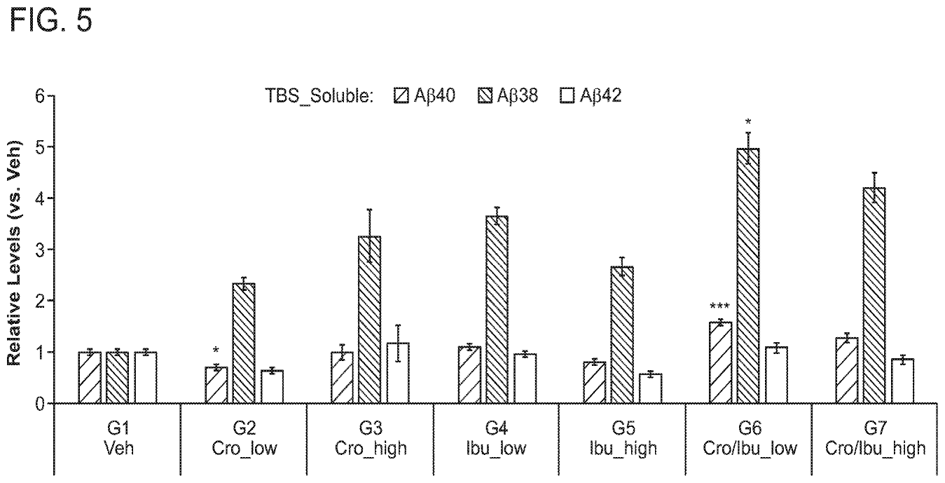

Cromolyn significantly affected the levels of brain TBS soluble Aβ and the ratios of Aβ (42:40). A-B. MSD (mess scale to measure Aβ 42,40,and 38) Aβ analyses were apply to brain TBS soluble samples. Differences in the Aβ levels and the ratios of Aβ (42:40) comparing the various treatment groups were analyzed. * p<0.05; **p <0.01, ***p <0.001, one-way ANOVA, Tukey's test; mean±SEM show that cromolyn and ibuprofen combination for the low and high dose higher relative level of Aβ 42/40 and ah higher Aβ 38 that is not implicated in plaque formation. FIG. 5 graphically illustrates the results of a one-way of the differences in the Aβ levels and the ratios of Aβ (42:40).

### Example 6: Effect of Cromolyn Sodium on Aβ42 Uptake in Microglial Cells

Confocal microscopy and ELISA assays were used to assess the effect of cromolyn and its derivative compounds on Aβ42 uptake in microglial cells. The BV2 microglial cell line, which was previously found to efficiently take up and degrade exogenously-added Aβ42, was used (Jiang, Q., et al. (2008) Neuron 58, 681-693; Mandrekar et al., 2009 J. Neurosci. 29, 4252-4262). The compounds were tested in naïve BV2 microglial cells to investigate whether they modulate Aβ uptake. The effect of compounds in BV2 cells stably expressing full-length human CD33 (BV2-CD33^{WT}) was assessed to explore whether they reverse CD33-mediated inhibition of Ap uptake (Griciuc et al., 2013 Neuron 78, 631-643).

The compound numbers, molecular weight and concentration of the stock solutions are summarized in Table 1. Cromolyn derivatives, C3 and C4, displayed lower solubility in DMSO in comparison to C1, C2, C5, C6, C7 and C8. Therefore, a 25 mM stock solutions for all the compounds except for C3 and C4 were prepared. The stock solutions for C3 and C4 were prepared at 5 mM and 7.5 mM, respectively. C1 is the parent compound—cromolyn di sodium.

TABLE 1
Summary of compounds tested in microglial cells

| Compound | | Stock Solution |
|---|---|---|
| Number | Compound Name | (mM) |
| C1 | Cromolyn Disodium | 25 |
| C2 | F-Cromolyn Diacid | 25 |
| C3 | ET-Cromolyn | 5 |
| C4 | F-ET-Cromolyn | 7.5 |
| C5 | Triol-Cromolyn | 25 |
| C6 | F-Triol-Cromolyn | 25 |
| C7 | Ac-Triol-Cromolyn | 25 |
| C8 | POM-Cromolyn | 25 |

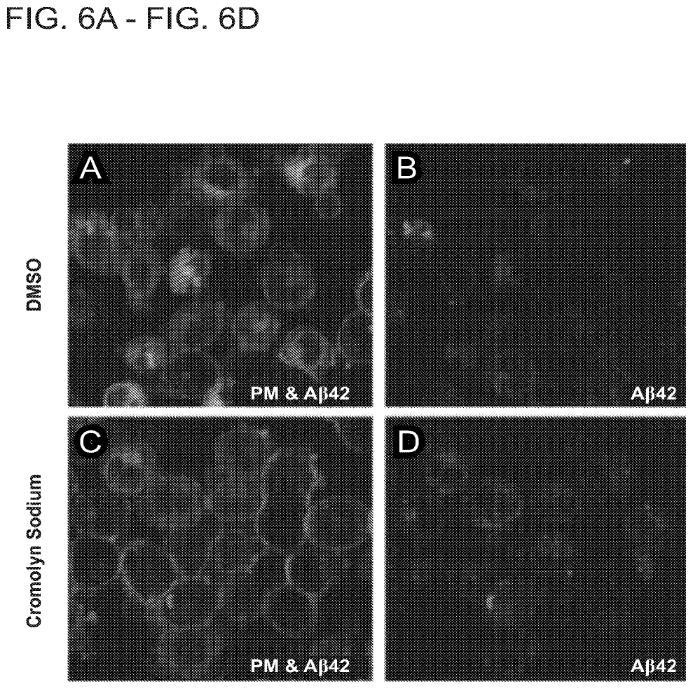

To investigate the effect of cromolyn sodium on Aβ42 uptake in microglial cells, naïve BV2 cells were treated with DMSO (control) or cromolyn at 500 μM for 16 hours. Afterwards, cells were washed with PBS and treated with DMSO or cromolyn in the presence of the fluorescently-tagged Aβ42 peptide (400 nM, red) for 2 hours. At the end of the treatment, the cells were washed and labeled them with a plasma membrane dye (green). Using confocal microscopy and the fluorescence signal in the red channel, the levels of intracellular Aβ42 peptide were quantified. All the quantifications were performed by a blind observer with the ImageJ software. Remarkably, cromolyn sodium led to increased uptake of Aβ42 in naïve BV2 microglial cells (FIG. 6A-FIG. 6D).

Furthermore, whether cromolyn sodium modulates Aβ42 uptake in naïve BV2 microglial cells was determined by using the ELISA assay. Additionally, whether cromolyn sodium leads to increased Aβ42 uptake levels in BV2 cells stably expressing full-length human CD33 (BV2-CD33^{WT}) was determined. To this purpose, both naïBV2 and BV2-CD33^{WT} cell lines were treated with DMSO (control) or cromolyn at different concentrations for 16 hours. Then, the cells were washed with PBS and treated with DMSO or cromolyn and soluble untagged Aβ42 peptide (400 nM) for 2 hours. The collected cell lysates were analyzed for Aβ42 uptake levels using the Aβ42-specific ELISA kit from Wako. The ELISA results were normalized to the protein concentration levels that were previously quantified using the BCA assay.

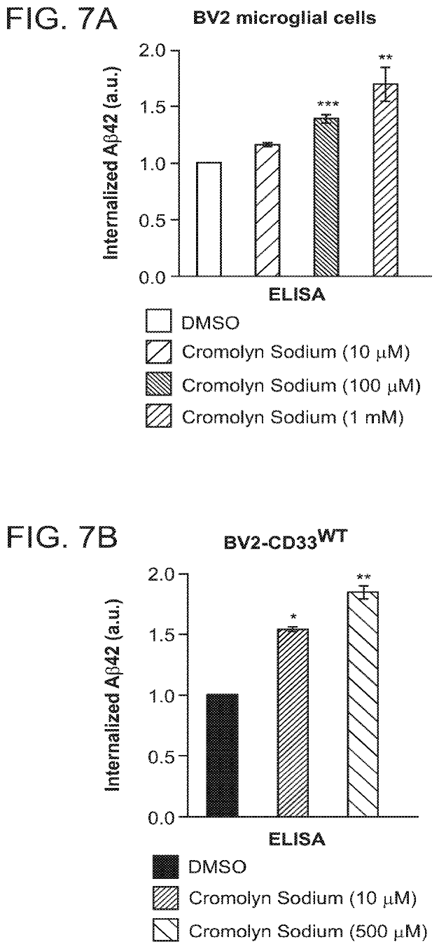

Cromolyn sodium led to increased Aβ42 uptake at 100 μM and 1 mM in naïBV2 microglial cells (FIG. 7A) and thus, confirmed the immunofluorescence results by ELISA assay. Cromolyn sodium also led to increased levels of internalized Aβ42 at 10 μM and 500 μM in BV2-CD33^{WT} cells (FIG. 7B, ELISA assay) and reversed CD33-mediated inhibition of Aβ42 uptake in microglial cells. In conclusion, treatment with cromolyn sodium showed a dose-dependent effect in modulating Aβ42 uptake levels in naïve BV2 and BV2-CD33^{WT} cell lines.

### Example 7: Effect of Cromolyn Derivatives on Aβ42 Uptake in Microglial Cells

To investigate the effect of cromolyn derivatives on Aβ42 uptake in microglia, naïBV2 or BV2-CD33^{WT} cells were plated in proliferating media. On the following day, cells were treated with DMSO (control) or the compounds at different concentrations in proliferating media for 3 hours. C1, C2, C5, C6, C7 and C8 were tested at 10, 50, 100 and 150 μM, while C3 and C4 were assessed at 5, 25, 50 and 75 μM due to solubility limit in DMSO. Afterwards, cells were washed with PBS and treated with DMSO or compounds in the presence of the untagged Aβ42 peptide (400 nM) in DMEM media for 2 hours. Compound toxicity was assessed in the media collected at the end of the treatment with CytoTox-ONE™ lactate dehydrogenase (LDH) assay. The remaining cells in the plate were washed with cold PBS and lysed with RIPA buffer supplemented with protease and phosphatase inhibitors. Protein concentrations in the lysate supernatants were determined using the Pierce™ BCA protein assay kit and 2-3 μg/well of total protein from each lysate was analyzed for Aβ42 uptake using the Aβ42 ELISA kit from Wako. Toxic compound concentrations were excluded from further studies.

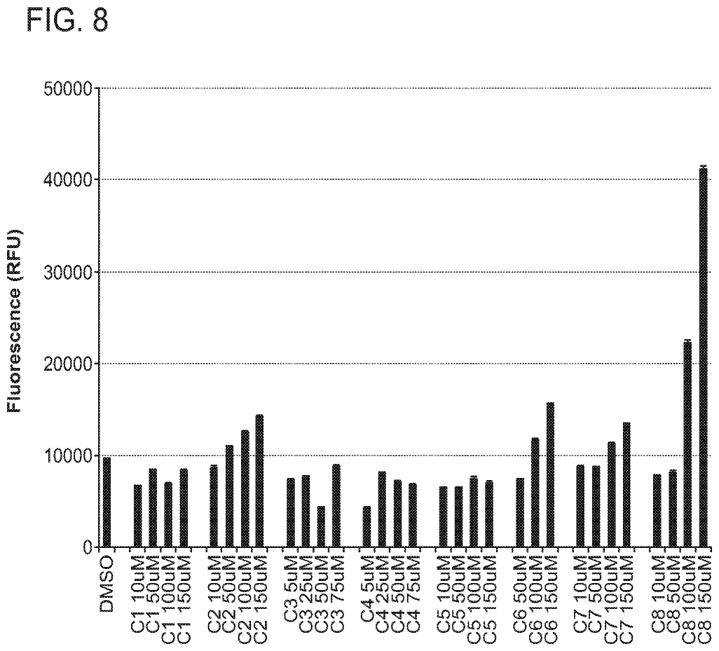

To investigate whether cromolyn derivatives induce cytotoxicity at higher doses, naïBV2 microglial cells were incubated with DMSO (vehicle) or cromolyn derivatives at different concentrations for 3 hours. The cells were then washed and incubated with DMSO or compounds and soluble untagged Aβ42 for additional 2 hours. Afterwards, the cell media was collected and measured LDH released by the damaged cells to identify the compounds that induce cytolysis. The LDH assay showed that the cromolyn derivative C8 is the only compound showing toxicity when tested at 100 and 150 μM (FIG. 8). Therefore, 100 and 150 μM concentrations for C8 were excluded from the Aβ42 uptake assays.

### Example 8: Modulation of Aβ42 Uptake in Microglial Cells by Cromolyn Derivatives

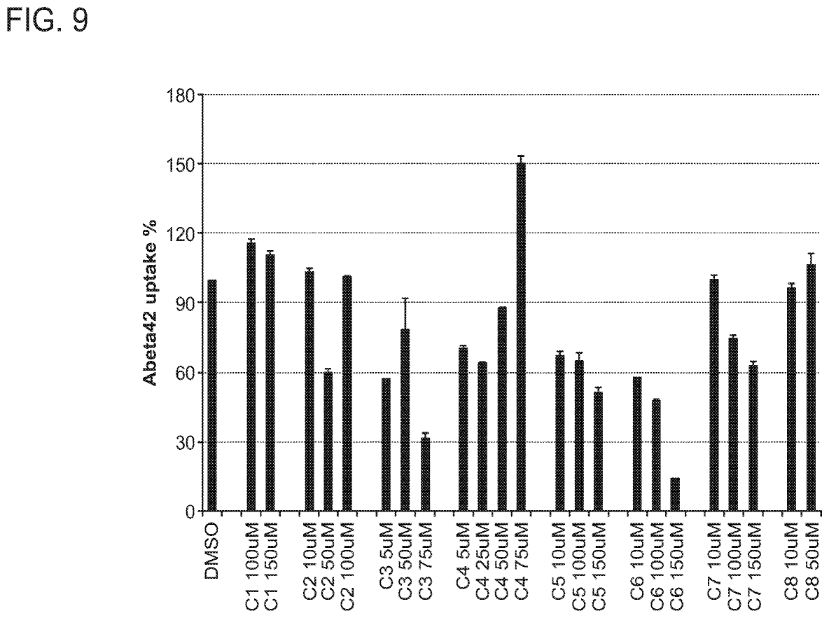

To test whether cromolyn derivatives modulate Aβ42 uptake, naïBV2 microglial cells were treated with DMSO (control) or cromolyn derivative compounds at different concentrations for 3 hours. Afterwards, the cells were washed and treated with DMSO or compounds in the presence of untagged Aβ42 peptide for 2 hours. At the end of the treatment, the cell lysates were collected. The analysis for intracellular Aβ₄₂ levels is performed using an Aβ42-specific ELISA kit. The parent compound C1 (cromolyn sodium) led to a modest increase of Aβ₄₂ uptake at 100 and 150 μM in BV2 cells. The C1 aliquot received with the other cromolyn derivatives displayed lower solubility in DMSO than the C1 aliquot that was sent to us the first time (without the cromolyn derivatives). Interestingly, the compound C6 led to a robust inhibition of Aβ42 uptake in BV2 microglial cells. Remarkably, the cromolyn derivative C4 led to an increased uptake of Aβ42 peptide at 75 μM in naïBV2 microglial cells (FIG. 9).

Further, whether cromolyn derivatives impact Aβ42 uptake and clearance in BV2-CD33^{WT} cells was determined by two independent sets of experiments. BV2-CD33^{WT} cells were treated with DMSO (control) or cromolyn derivatives at different concentrations ranging between 5 and 150 μM.

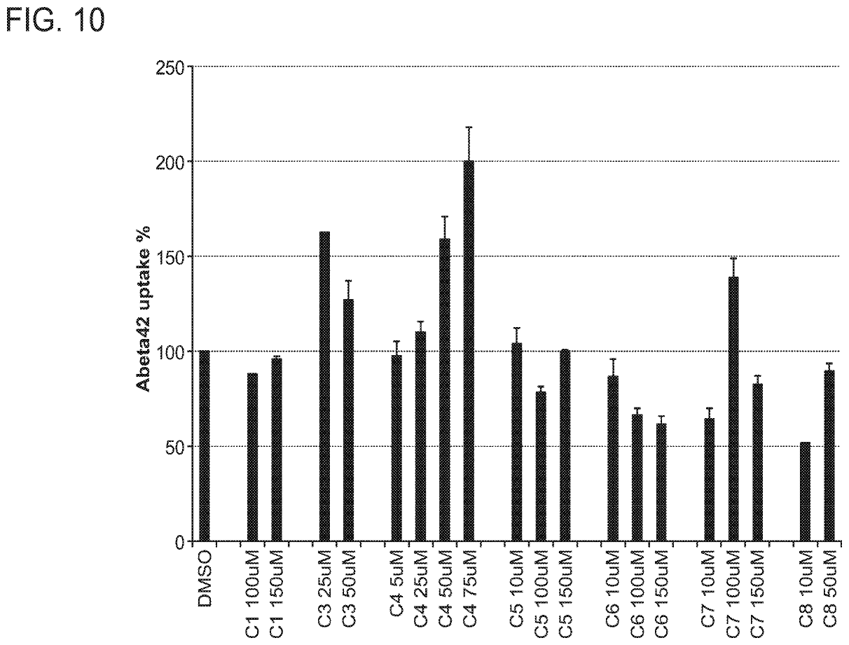

In the first set of experiments, the cromolyn derivatives C1 and C3-8 were tested. The compound C2 was tested with other cromolyn derivatives in the second set of experiments. Treatment with the compound C4 at 75 μM resulted in a two-fold increase in Aβ42 uptake in comparison to DMSO treatment and displayed a dose-dependent effect at 50 μM (FIG. 10). Using the GraphPad Prism 7 Software, the IC₅₀ for C4 was 54.7 μM in BV2-CD33^{WT} cells. The compound C6 exhibits a dose-dependent effect in mediating inhibition of Aβ42 uptake in BV2-CD33^{WT} cells when compared to DMSO treatment.

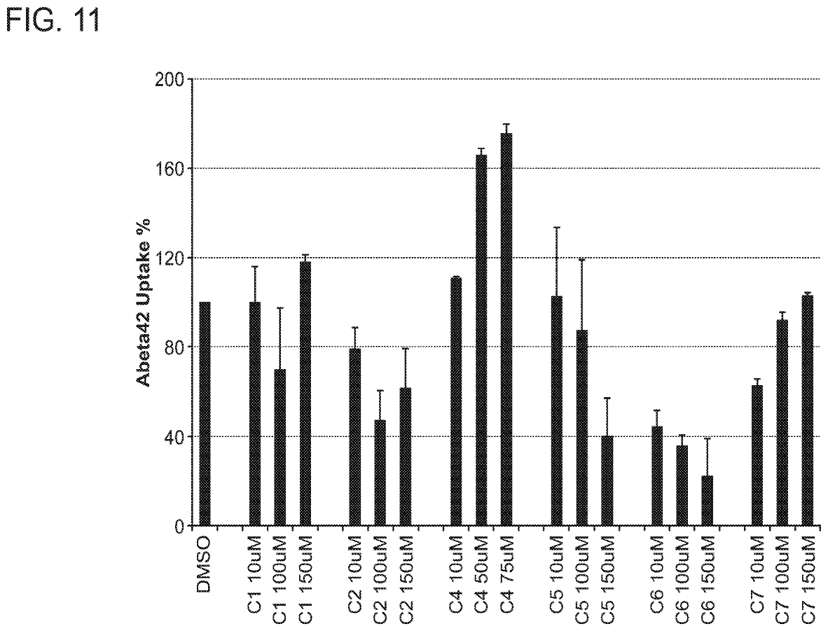

In the second set of experiments, the cromolyn derivatives C1, C2, and C4-7 in BV2-CD33^{WT} cells was tested. These results confirmed prior results that the compound C4 was the most effective in increasing the Aβ42 uptake at 75 μM and displayed a dose-dependent effect at lower concentrations when compared to DMSO treatment (FIG. 11). Thus, these results suggest that the compound C4 led to increased Aβ42 uptake levels in BV2-CD33^{WT} cells and reversed the CD33-mediated inhibition of Aβ uptake and clearance (FIGS. 10 and 11).

These results suggest that the cromolyn derivative C4 induced microglial uptake and clearance of Aβ42 and enhanced skewing of microglial cells from the neurotoxic/pro-inflammatory towards neuroprotective/pro-phagocytic activation phenotype.

1. A method of treating a neuron inflammation condition in a patient in need thereof comprising administering to the patient a therapeutically effective amount of at least one compound selected from: 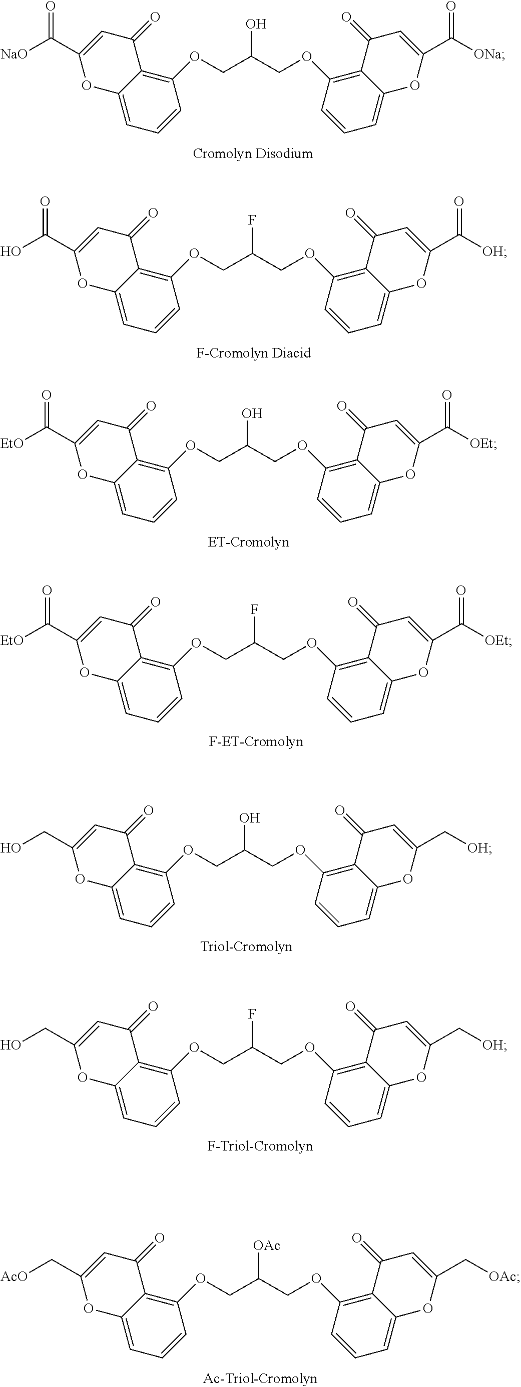 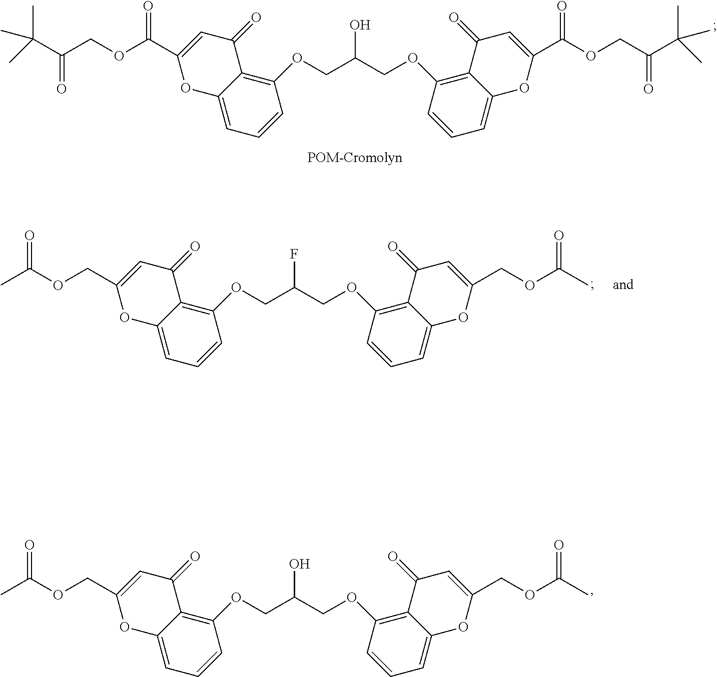
wherein the neuron inflammation condition is amyotrophic lateral sclerosis (ALS), Huntington's Disease, Parkinson's disease (PD), ischemic stroke, or a condition associated with prion disease.

2. (canceled)

3. The method of claim 1, wherein the neuron inflammation condition is ALS.

4. (canceled)

5. The method of claim 1, wherein the neuron inflammation is Huntington's Disease.

6. The method in claim 1, wherein the neuron inflammation is Parkinson's disease.

7. The method of claim 1, wherein the neuron inflammation condition is ischemic stroke.

8. The method of claim 1, wherein the neuron inflammation condition is associated with prion disease.

9. The method of claim 3, wherein the compound is administered via IP and/or IV.

10. The method of claim 1, wherein the compound is administered transdermally.

11. The method of claim 1, wherein the compound is administered by inhalation.

12. The method of claim 1, wherein the compound is administered at a dose between about 1 mg and about 1000 mg per day.

13. The method of claim 1, where in the compound is administered at a dose of about 10, about 20, about 30, about 50, about 100, or about 500 mg per day.

14. The method of claim 3, further comprising co-administering a second compound selected from CD4+; siRNA; miRNA that ameliorates ALS; glial morphology modifier; SOD1 control; and Riluzole.

15. The method of claim 3, further comprising co-administering a second compound selected from an anti-aggregation drug and a targeting drug for AD.

16-18. (canceled)

19. The method of claim 3, further comprising co-administering a second compound selected from an antibody targeting drug that ameliorates ALS.

20. The method of claim 3, further comprising co-administering a second compound selected from an anti-inflammatory targeting drug that ameliorates ALS.

21. The method of claim 1, further comprising co-administering a second compound selected from a targeting drug that ameliorates neurodegeneration associated with amyloidosis or tauopathies.

22. The method of claim 6, further comprising co-administering a second compound selected from an alpha synuclein targeting drug that ameliorates PD and a Parkinson's targeting drug that ameliorates PD.
